SEQUENCE LISTING (2 sequences)

SEQ ID NO: 1 - DNA, 1008 bp, Homo sapiens
atggggtgcctgctgtttctgctgctctgggcgctcctccaggcttggggaagcgctgaagtcccgcaaaggcttttccccctccgctgcctccagatctcgtccttcgccaatagcagctggacgcgcaccgacggcttggcgtggctgggggagctgcagacgcacagctggagcaacgactcggacaccgtccgctctctgaagccttggtcccagggcacgttcagcgaccagcagtgggagacgctgcagcatatatttcgggtttatcgaagcagcttcaccagggacgtgaaggaattcgccaaaatgctacgcttatcctatcccttggagctccaggtgtccgctggctgtgaggtgcaccctgggaacgcctcaaataacttcttccatgtagcatttcaaggaaaagatatcctgagtttccaaggaacttcttgggagccaacccaagaggccccactttgggtaaacttggccattcaagtgctcaaccaggacaagtggacgagggaaacagtgcagtggctccttaatggcacctgcccccaatttgtcagtggcctccttgagtcagggaagtcggaactgaagaagcaagtgaagcccaaggcctggctgtcccgtggccccagtcctggccctggccgtctgctgctggtgtgccatgtctcaggattctacccaaagcctgtatgggtgaagtggatgcggggtgagcaggagcagcagggcactcagccaggggacatcctgcccaatgctgacgagacatggtatctccgagcaaccctggatgtggtggctggggaggcagctggcctgtcctgtcgggtgaagcacagcagtctagagggccaggacatcgtcctctactggggtgggagctacacctccatgggcttgattgccttggcagtcctggcgtgcttgctgttcctcctcattgtgggctttacctcccggtttaagaggcaaacttcctatcagggcgtcctgtga

SEQ ID NO: 2 - protein, 335 aa, Homo sapiens
MGCLLFLLLWALLQAWGSAEVPQRLFPLRCLQISSFANSSWTRTDGLAWLGELQTHSWSNDSDTVRSLKPWSQGTFSDQQWETLQHIFRVYRSSFTRDVKEFAKMLRLSYPLELQVSAGCEVHPGNASNNFFHVAFQGKDILSFQGTSWEPTQEAPLWVNLAIQVLNQDKWTRETVQWLLNGTCPQFVSGLLESGKSELKKQVKPKAWLSRGPSPGPGRLLLVCHVSGFYPKPVWVKWMRGEQEQQGTQPGDILPNADETWYLRATLDVVAGEAAGLSCRVKHSSLEGQDIVLYWGGSYTSMGLIALAVLACLLFLLIVGFTSRFKRQTSYQGVL

(19) United States
(12) Patent Application Publication
(10) Pub. No.: US 2013/0344095 A1
(43) Pub. Date: Dec. 26, 2013

(54) METHOD OF USING CD1D OVER-EXPRESSION IN HUMAN DENDRITIC CELLS TO ENHANCE CD8+ T CELL-BASED AND INVARIANT NATURAL KILLER T CELL-BASED ANTITUMOR IMMUNITY

(71) Applicants: Shu Wang, Singapore (SG); Jieming Zeng, Singapore (SG)

(72) Inventors: Shu Wang, Singapore (SG); Jieming Zeng, Singapore (SG)

(21) Appl. No.: 14/004,413

(22) PCT Filed: Mar. 9, 2012
(86) PCT No.: PCT/SG2012/000080

§ 371 Date: Sep. 10, 2013

(30) Foreign Application Priority Data
Mar. 10, 2011 (SG) 201101737-3

(51) Int. Cl. C12N 5/0784

(52) U.S. Cl. CPC C12N 5/0639
USPC 424/184.1; 435/320.1; 435/69.1

(57) ABSTRACT

The manipulation of human dendritic cells (DCs) to induce potent anti-tumor immunity remains an essential subject of study. Here we report that the overexpression of CD1d in human DCs can enhance the priming of naïve CD8+ T cells against tumor antigen. We showed that CD1d can be overexpressed in the human DCs using baculoviral vector carrying the CD1d gene. This CD1d-overexpression is functional as demonstrated by the increased expansion of invariant natural killer T (iNKT) cells while using these modified DCs to present α-galactosylceramide (α-GC). Pulsed with tumor antigenic peptide, these CD1d-overexpressing human DCs showed enhanced capability to prime naïve CD8+ T cells. CD1d-overexpressing human DCs also induced a pro-inflammatory cytokine profile that may favor the priming. Moreover, this CD1d-overexpression strategy can be extrapolated to monocyte-derived human DCs. Therefore, our study suggest that overexpression of CD1d in human DCs may provide a novel strategy to enhance DC immunogenicity and the possible translation into human cancer immunotherapy.

### CROSS-REFERENCE TO RELATED APPLICATION

This application claims the benefit of Singapore Patent Application No. 201101737-3 filed on 10 Mar. 2011, the entire contents of which are incorporated herein by reference.

### FIELD

The invention relates to methods and vectors suitable for use in cancer vaccine and/or treatment

### BACKGROUND ART

Cancer is one of the main diseases of current times causing 13% of all deaths globally. The term cancer vaccine may refer to a vaccine that aims to either prevent infections with cancer-causing viruses, preventing the development of cancer in certain high risk individuals (vaccines against infectious agents) or treats existing cancer (therapeutic vaccines). One approach to cancer vaccination is to separate proteins from cancer cells and immunize cancer patients against those proteins, in the hope of stimulating an immune reaction that would kill the cancer cells. Therapeutic cancer vaccines seek to target an antigen specific to the tumor and distinct from proteins of the vaccine. It is very difficult to select an appropriate adjuvant to simulate an immune response as cancer cells have developed within the person's body in the presence of the immune system.

Therapeutic cancer vaccines are being developed for the treatment of breast, lung, colon, skin, kidney, prostate, and other cancers. Most clinical trials investigating a therapeutic cancer vaccine have failed or had very modest responses by standardized oncologic assessment criteria (RESIST). There have been two therapeutic cancer vaccines approved, one in Russia for the treatment of Kidney cancer and one in the U.S.A. for late stage prostate cancer. Both of these rely on autologous cell therapeutics and must be prepared specifically for each patient.

Dendritic cells (DCs), known as professional antigen-presenting cells (APCs) are one of the attractive manipulation targets in cancer immunotherapy due to their indispensable ability to initiate and regulate anti-tumor T cell immunity.¹ With approval of the first DC-based vaccine, Sipuleucel-T (APC8015, trade name Provenge), manufactured by Dendreon Corporation, autologous DC-based therapy is being established as a new modality for cancer treatment (2). However, the preparation of autologous cell therapeutics is expensive for patients and technically demanding for clinicians, not to mention the difficulty for large-scale industrial production (2). Such patient-customized vaccine is faced with inherent problems such as limited DC number, high variability in DC quality and function, serious logistic issue and high production cost. From industrial standpoint, “Herculean effort” is required to produce such an autologous cell therapy on large-scale (2).Great numbers of DC cells are needed because of the high variability in DC quality and function as the current process results in only an average percentage of the adapted DC cells able to stimulate a response. Hence, a more effective adjuvant is required.

It is gradually appreciated that the cross-talk between DCs and innate lymphocytes may play an important role at the initiation of adaptive immune response (6). Invariant natural killer T (iNKT) cells are innate lymphocytes evolved to recognized lipid or glycolipid antigens presented by CD1d, a non-classical class I-like major histocompatibility complex (MHC) molecule (7). iNKT cells represent a unique subset of T-lymphocytes that they express an invariant T-cell receptor (TCR) a chain (Vα14-Jα18 in mice and Vα24-Jα18 in humans) paired with a semi-invariant TCR β chain (Vβ2, Vβ7 or Vβ8.2 in mice and Vβ11 in humans) (7). Although iNKT cells possess some similarities with natural killer (NK) cells and cytotoxic T lymphocytes (CTLs) in terms of functions and gene expression patterns,(8,9) they may only kill CD1d-positive leukemic cell lines(10,11) or tumor cell lines that have been transfected with CD1d and pulsed with α-galactosylceramide (α-GC)(12). Functionally, iNKT cells more closely resemble that of helper T cells (8,9) and have a memory-activated phenotype (7). One prominent feature of iNKT cells is that they may release copious amounts and wide variety of cytokines quickly after activation (7, 14). Earlier studies have failed to enhance T cell priming by using the α-GC- and OVA peptide-loaded DCs and they speculated that it may be due to the elimination of such DCs by the activated iNKT cells or specific CD8+ T cells (35). Currently, most studies on iNKT cells have used a potent agonist α-GC as stimulus. However, the nature, strength and context of stimulus as well as the types and activation status of APCs may have profound impacts on iNKT cell activation. As demonstrated in mouse studies, systemic administration of α-GC could result in uncontrolled iNKT cell activation and cause side effects such as “cytokine storm” or iNKT cell anergy (13, 14). The induction of iNKT cell response by α-GC-loaded DCs could reduce these undesired effects (15), but the stimulated iNKT cells can kill these DCs (16, 17) and render them incapable of initiating a specific protective immunity. Hence, one strategy to manipulate iNKT cell response is to use synthetic iNKT cell-agonists (18). Through structure-guided design, these novel iNKT-cell agonists may minimize the side effects associated with iNKT cell overstimulation by strong agonists (e.g. α-GC), while inducing differential cytokine production. A pure Th1-like or Th2-like response can be generated by these synthetic α-GC analogs, which may be attributed to their different affinity to the iNKT TCRs, although the detailed mechanisms remain unclear.

Cancer immunotherapy offers great promise in the fight against cancer, especially for persistent malignant cells. The major strength of the approach lies in stimulating the patient's own immune cells to elicit immune responses against tumors. DCs are the most efficient antigen-presenting cells for priming naive T cells into effectors that specifically kill tumors. However, there is an acute need to improve the priming ability in the current DC-based approaches.

### SUMMARY

In this study, we examined, for the first time, whether the enforced up-regulation of CD1d in DCs can be used to exploit the iNKT cell adjuvant activity and assist the priming of CD8+ T cells against tumor antigen.

Accordingly a first aspect of the invention is an expression vector comprising a polynucleotide that encodes a CD1d operably linked to a regulatory sequence capable of up-regulating expression of said polynucleotide in a host cell.

Preferably the polynucleotide encodes a CD1d comprising SEQ ID NO. 1.

In one embodiment the expression vector is for use in the manufacture of a vaccine for the treatment of a cancer.

Preferably the expression vector is able to stably or transiently express the CD1d in dendritic cells or their progenitors. In one embodiment the vector is a baculoviral vector carrying the CD polypeptide

Preferably the host cell is a dendritic cell or a progenitor cell of dendritic cell such as a human embryonic stem cell or a human induced pluripotent stem cell. In one embodiment the dendritic cell is derived from a human embryonic stem cell or a human pluripotent stem cell.

Another aspect of the invention comprises a method of manufacturing a vaccine for enhancing an immune response to a dendritic cell comprising the steps of: a) culturing the dendritic cells in vitro; and b) introducing a polynucleotide that encodes a CD and overexpresses the CD in the dentritic cell.

Preferably the polynucleotide comprises SEQ ID NO. 1.

Preferably the dendritic cell is generated from a human embryonic stem cell or a human pluripotent stem cell.

Preferably the polynucleotide is introduced to the denritic cell in culture by the expression vector of the invention.

Another aspect of the invention comprises a method of enhancing an immune response to a dendritic cell for treating a patient in need of a cancer treatment comprising the steps of: a) culturing the dendritic cells in vitro; b) introducing a polynucleotide that encodes a CD1d and overexpresses the CD1d in the dentritic cell; and c) introducing the CD1d-overexpressing denritic cell to the patient in need of a cancer treatment.

In one embodiment the dendritic cell is isolated from the patient.

In another embodiment the dendritic cell is generated from a human embryonic stem cell or a human pluripotent stem cell.

Preferably the polynucleotide comprises SEQ ID NO. 1.

Another aspect of the invention comprises use of the expression vector of the invention for the preparation of a medicament for the treatment of a cancer.

Another aspect of the invention comprises a cancer vaccine comprising a CD1d-overexpressing dendritic cell having upregulated expression of a CD1d polynucleotide.

Preferably the dendritic cell is selected from a group consisting of a cell isolated from an individual having cancer and a cell generated from a human embryonic stem cell or a human pluripotent stem cell.

Preferably the CD1d polynucleotide of the vaccine comprises SEQ ID NO. 1.

### BRIEF DESCRIPTION OF DRAWINGS

FIG. 1. Generation of human DCs from hESCs using a three-step culture protocol. (A-D) Morphological changes of the cells during the differentiation process. (A) Differentiated HES-1 cells grown on OP9 cells for 9 days. (B) Harvested HES-1/OP9 coculture grown in suspension with GM-CSF for 10 days. (C) HES-1/OP9 coculture further differentiated in suspension with GM-CSF and IL-4 for 8 days. (D) Morphology of the HES-1-derived DCs cultured on normal cell culture dish. (E) Phenotype of differentiated H1, H9 and HES-1 after the three-step culture as analyzed by flow cytometry. Histograms are showing the staining by antibodies against indicated markers (red line) and their isotype controls (black line). The percentages of the positive cells are indicated.

FIG. 2. Transduction of purified H1.DCs by baculoviral vectors. (A-C) Morphology of the cells before and after purification. To purify H1.DCs from the differentiated H1, the pre-sort population (A) was stained with anti-CD209 antibody and sorted by flow cytometer to generate a homogenous post-sort population (B). These post-sort H1.DCs grew as attached cells on normal cell culture surface (C). (D) Fluorescence image of these H1.DCs 2 days after transduction by BV-CMV.eGFP at MOI of 100. (E) Phenotypic analysis of these H1.DCs 2 days after transduction by BV-PacPAK6. Histograms are showing the staining by antibodies against indicated markers (red line) and their isotype controls (black line). The numbers are showing the change of mean fluorescence intensity (ΔMFI). (F) Allostimulatory function of H1.DCs after baculoviral transduction. The H1.DCs were transduced by BV-PacPAK6 at the indicated MOIs. Two days later, the DCs were incubated with CFSE-labeled pan-T cells at the indicated ratios. PHA was used to stimulate pan-T cells as positive control. After incubation of 5 days, the cells were stained with APC mouse anti-human CD3 antibody and the T cell proliferation was measured by CFSE dilution using flow cytometer. The numbers in the histograms are showing the percentage of divided T cells.

FIG. 3. CD1d-overexpression in H1.DCs using baculoviral vectors is functional in promoting human iNKT cell expansion. (A) The expression of CD1d on H1.DCs 2 days after transduction with BV-PacPAK6 (BV) or BV-CMV.CD1d-WPRE (BVCD1d) at the indicated MOIs was analyzed using flow cytometry. Histograms are showing the staining by antibody against CD1d (red line) and the isotype control (black line). The numbers are showing the percentage of the positive cells. (B-E) In vitro expansion of iNKT cells induced by CD1d-overexpressing H1.DCs. Two days after baculoviral transduction, H1.DCs were co-cultured with (B) human PBLs or pan-T cells in the presence of α-GC or with (C) pan-T cells in the presence or absence of α-GC for 7 days. iNKT cells were then analyzed by flow cytometry. Dot plots and the percentages of the iNKT cells in total T cells are shown. (D) The comparison of iNKT cell expansion induced by autologous moDCs and CD1d-overexpressing H1.DCs after 7 days coculture with PBLs. (E) The expansion of iNKT cells induced by α-GC-pulsed baculovirus-transduced CD1d-overexpressing H1.DCs.

FIG. 4. CD1d-overexpression in H1.DCs enhances priming of CD8+ T cells against tumor antigen. (A-B) The induction of tumor antigen-specific T cell response by CD1d-overexpressing H1.DCs. The H1.DCs were first transduced by baculoviral vectors and then pulsed with α-GC for 24 h one day after transduction. Two days after transduction, the DCs were pulsed with MART-1 peptide for 4 h and used to stimulate HLA A2+PBLs. After coculture of 9 days, the samples were stained with anti-CD3, anti-CD8 and A*0201/ELAGIGILTV Pentamer and analyzed by flow cytometer. The numbers in the dot plots indicate the percentage of Pentamer+ CD8+ cells in CD3+ population. (C) The cocultures were restimulated with MART-1 peptide-pulsed H1.DCs on day 9 of coculture and analyzed one week after restimulation. To restimulate the coculture primed by autologous moDCs, MART-1 peptide-pulsed autologous moDCs were used. (D) To measure antigen-specific CTL activity, the cocultures were stimulated for the third time on day 7 after second stimulation with MART-1 peptide-pulsed H1.DCs and used as effectors for lysis of tumor cells one week later.

FIG. 5. CD1d-overexpression strategy can be applied to moDCs for enhancing T cell priming. (A) The expression of CD1d on moDCs 2 days after transduction with BV-PacPAK6 (BV) or BV-CMV.CD1d-WPRE (BVCD1d) at MOI of 100 was analyzed using flow cytometry. Histograms are showing the staining by antibody against CD1d (red line) and the isotype control (black line). The numbers are showing the percentage of the positive cells. (B) The expansion of iNKT cells induced by α-GC-pulsed CD1d-overexpressing autologous moDCs. Dot plots and the percentages of the iNKT cells in total T cells are shown. (C-D) The induction of tumor antigen-specific T cell response by CD1d-overexpressing autologous moDCs. The moDCs were first transduced by baculoviral vectors. Two days after transduction, the moDCs were pulsed with MART-1 peptide for 4 h and used to stimulate HLA A2+PBLs. After coculture of 9 days, the samples were stained and analyzed by flow cytometer. The numbers in the dot plots indicate the percentage of Pentamer+ CD8+ cells in CD3+ population.

FIG. 6. Enhancing T cell priming by CD1d-overexpression depends on DC and iNKT cell interaction. (A) The reduction of T cell priming ability of CD1d-overexpressing human DCs by blocking CD1d with anti-CD1d antibody. The H1.DCs or moDCs were first transduced by BV-CMV.CD1d-WPRE (BVCD1d). Two days after transduction, the DCs were pulsed with MART-1 peptide for 4 h and blocked by anti-CD1d blocking antibody or its isotype control before used to stimulate HLA A2+PBLs. After co-culture of 9 days, the samples were stained and analyzed by flow cytometer. The numbers in the dot plots indicate the percentage of Pentamer+ CD8+ cells in CD3+ population. (B-C) Cytokine production during the co-culture of CD1d-overexpresing H1.DCs and PBLs. In B, H1.DCs were transduced with BV or BVCD1d and incubated with PBLs two days after transduction. In C, CD1d-overexpresing H1.DCs were pretreated with anti-CD1d blocking antibody or its isotype control before co-culturing with PBLs. The supernatants were collected after co-culture for 3 days and cytokine concentration was measured by CBA assay. Results represent mean±SD, n=4. The p values are derived from two-sided Student's t test. (D) Inhibition of T cell priming ability of CD1d-overexpressing human DCs by glycolipid biosynthesis inhibitors. The DCs were first transduced with BVCD1d. One day after transduction, the DCs were treated with PPMP or NB-DGJ for 24 hours. Two days after transduction, the MART-1 peptide-pulsed DCs were used to stimulate HLA-A2+PBLs. The percentages of Pentamer+ CD8+ cells in CD3+ population after 9-day co-culture are indicated in the representative contour plots. (E) Comparison of DC surface marker expression after transduction with BV and BVCD1d. The DCs were transduced with BV or BVCD1d, stained and analyzed by flow cytometer two days after transduction. Histograms show the staining by specific antibodies against indicated markers and the isotype control.

### DETAILED DESCRIPTION

Regulation of CD1d expression and their presentation of either foreign- or self-antigens on the DC surface may present a novel strategy to affect the iNKT cell activation and its immunological outcome. In this present study, we investigate for the first time the effect of CD1d-overexpression in human DCs on the activation of iNKT cells and the immunological effect on the priming of naïve CD8+ T cells against tumor antigen. Starting with the human embryonic stem cell (hESC)-derived DCs (hESC-DCs) that providing us a standardized and unlimited source of human DCs, we show that CD1d can be overexpressed in these hESC-DCs using baculoviral vectors carrying the CD1d gene. Based on these platforms, we further show that baculovirus-mediated CD1d-overexpression is functional in promoting iNKT cell expansion and enhances the immunogenicity function of these modified DCs. Moreover, this CD1d-overexpression strategy can be extended to monocyte-derived human DCs.

Here we report that the overexpression of CD1d in human DCs can enhance the priming of naïve CD8+ T cells against tumor antigen. By using the human embryonic stem cell (hESC)-derived DCs as a standardized source of human DCs, we showed that CD1d can be overexpressed in these human DCs using a baculoviral vector carrying the CD gene. The overexpressed CD molecules are functional as demonstrated by increased expansion of invariant natural killer T (iNKT) cells when the modified DCs were used to present α-galactosylceramide (α-GC). Pulsed with a tumor antigenic peptide, these CD1d-overexpressing human DCs showed enhanced capability to prime naïve CD8+ T cells. The priming ability was reduced after incubation of CD1d-overexpressing human DCs with the anti-CD1d antibody, suggesting the importance of the interaction between DCs and iNKT cells for the enhanced priming. CD1d-overexpressing human DCs also induced a pro-inflammatory cytokine profile that may favour the priming. Moreover, this CD1d-overexpression strategy can be extrapolated to monocyte-derived human DCs. Therefore, overexpression of CD1d in human DCs provides a novel strategy to enhance DC immunogenicity that can possibly be adopted for human cancer immunotherapy.

Without being limited to any theory we reason that one possible explanation for the negative results observed in earlier human cell studies that tried to utilize iNKT cell adjuvant activity is the use of α-GC. This potent agonist may overstimulate the human iNKT cells and result in elimination of the α-GC-loaded DCs. Therefore, overstimulation of iNKT cells could certainly pose a problem for using the α-GC and tumor antigen double-loaded DC strategy to induce anti-tumor immunity, indicating that optimal stimulation of iNKT cells could be critical. The strength of such optimal stimulation may lie between that provided by the physiological level of endogenous iNKT ligands and that rendered by the potent agonist like α-GC. Hence, the up-regulation of antigen/CD1d complexes on the DC surface may provide such optimal strength to activate iNKT cells. To this end, we propose a novel strategy that employs enforced overexpression of CD1d on human DCs to utilize the human iNKT cell adjuvant activity. We demonstrated that such CD1d-overexpressing human DCs were effective in promoting tumor antigen-specific CTL response.

Throughout the description the following terms have the explained meaning or the meaning that would be understood by a person having ordinary skill in the art. DC, dendritic cell; hESC, human embryonic stem cells; hESC-DC, human embryonic stem cell-derived dendritic cell; iNKT cell, invariant natural killer T cell; α-GC, α-galactosylceramide; BV, baculovirus carrying BacPAK6 viral gene; BVCD1d, baculovirus carrying CD1d gene; MOI, multiplicity of infection; CFSE, carboxyfluorescein diacetate succinimidyl ester; MART-1, melanoma antigen recognized by T cell 1; PPMP, DL-threo-1-Phenyl-2-palmitoylamino-3-morpholino-1-propanol; NB-DGJ, N-(n-Butyl)deoxygalactonojirimycin; RLU, relative light unit; CBA, cytometric bead array; H1.DC, H1-derived dendritic cell.

### Vectors

A first aspect of the invention is an expression vector comprising a polynucleotide that encodes a CD1d operably linked to a regulatory sequence capable of up-regulating expression of said polynucleotide in a host cell.

Preferably the polynucleotide encodes a CD1d comprising SEQ ID NO. 1.

Preferably the expression vector is for use in the manufacture of a vaccine for the treatment of a cancer.

Preferably the expression vector is a baculoviral vector carrying the CD1d polynucleotide or any other vectors that may stably or transiently express the CD1d in dendritic cells or their progenitors.

The present invention also provides a vector comprising a polynucleotide of the invention, for example an expression vector comprising a polynucleotide of the invention, operably linked to regulatory sequences capable of directing expression of said polynucleotide in a host cell.

Any CD1d nucleic acid specimen, in purified or non-purified form, can be utilised as the starting nucleic acid or acids.

PCR is one such process that may be used to amplify isolated CD1d sequences. This technique may amplify, for example, DNA or RNA, including messenger RNA, wherein DNA or RNA may be single stranded or double stranded. In the event that RNA is to be used as a template, enzymes, and/or conditions optimal for reverse transcribing the template to DNA would be utilized. In addition, a DNA-RNA hybrid that contains one strand of each may be utilized. A mixture of nucleic acids may also be employed, or the nucleic acids produced in a previous amplification reaction described herein, using the same or different primers may be so utilised.

The specific nucleic acid sequence to be amplified, may be a fraction of a nucleic acid or can be present initially as a discrete nucleic acid, so that the specific sequence constitutes the entire nucleic acid. It is not necessary that the sequence to be amplified is present initially in a pure form; it may be a minor fraction of a complex mixture, such as contained in whole human DNA.

DNA utilized herein may be extracted from a body sample, such as blood, tissue material, lung tissue and the like by a variety of techniques known in the art. If the extracted sample has not been purified, it may be treated before amplification with an amount of a reagent effective to open the cells, or animal cell membranes of the sample, and to expose and/or separate the strand(s) of the nucleic acid(s). This lysing and nucleic acid denaturing step to expose and separate the strands will allow amplification to occur much more readily.

The deoxyribonucleotide triphosphates dATP, dCTP, dGTP and dTTP are added to the synthesis mixture, either separately or together with the primers, in adequate amounts and the resulting solution is heated to about 90 degrees-100 degrees C. from about 1 to 10 minutes, preferably from 1 to 4 minutes. After this heating period, the solution is allowed to cool, which is preferable for the primer hybridization. To the cooled mixture is added an appropriate agent for effecting the primer extension reaction (called herein “agent for polymerization”), and the reaction is allowed to occur under conditions known in the art. The agent for polymerization may also be added together with the other reagents if it is heat stable. This synthesis (or amplification) reaction may occur at room temperature up to a temperature above which the agent for polymerization no longer functions. Thus, for example, if DNA polymerase is used as the agent, the temperature is generally no greater than about 40 degree C. Most conveniently the reaction occurs at room temperature.

Primers direct amplification of a target polynucleotide (eg CD1d). Primers used should be of sufficient length and appropriate sequence to provide initiation of polyrmerisation. Environmental conditions conducive to synthesis include the presence of nucleoside triphosphates and an agent for polymerisation, such as DNA polymerase, and a suitable temperature and pH.

Primers are preferably single stranded for maximum efficiency in amplification, but may be double stranded. If double stranded, primers may be first treated to separate the strands before being used to prepare extension products. Primers should be sufficiently long to prime the synthesis of CD1d of the invention, into extension products in the presence of the inducing agent for polymerization. The exact length of a primer will depend on many factors, including temperature, buffer, and nucleotide composition. Oligonucleotide primers will typically contain 12-20 or more nucleotides, although they may contain fewer nucleotides.

Primers should be designed to be substantially complementary to each strand of the CD1d genomic gene sequence. This means that the primers must be sufficiently complementary to hybridise with their respective strands under conditions that allow the agent for polymerisation to perform. In other words, the primers should have sufficient complementarity with the 5′ and 3′ sequences flanking the mutation to hybridise therewith and permit amplification of the CD genomic gene sequence.

Oligonucleotide primers of the invention employed in the PCR amplification process that is an enzymatic chain reaction that produces exponential quantities of CD1d gene sequence relative to the number of reaction steps involved. Typically, one primer will be complementary to the negative (−) strand of the CD1d gene sequence and the other is complementary to the positive (+) strand. Annealing the primers to denatured nucleic acid followed by extension with an enzyme, such as the large fragment of DNA polymerase I (Klenow) and nucleotides, results in newly synthesised + and − strands containing the target CD1d gene sequence. Because these newly synthesized sequences are also templates, repeated cycles of denaturing, primer annealing, and extension results in exponential production of the region (i.e., the CD1d gene sequence) defined by the primers. The product of the chain reaction is a discreet nucleic acid duplex with termini corresponding to the ends of the specific primers employed.

Oligonucleotide primers may be prepared using any suitable method, such as conventional phosphotriester and phosphodiester methods or automated embodiments thereof. In one such automated embodiment, diethylphosphoramidites are used as starting materials and may be synthesized as known in the art.

The agent for polymerisation may be any compound or system which will function to accomplish the synthesis of primer extension products, including enzymes. Suitable enzymes for this purpose include, for example, E. coli DNA polymerase I, Klenow fragment of E. coli DNA polymerase, polymerase muteins, reverse transcriptase, other enzymes, including heat-stable enzymes (ie, those enzymes which perform primer extension after being subjected to temperatures sufficiently elevated to cause denaturation), such as Taq polymerase. Suitable enzyme will facilitate combination of the nucleotides in the proper manner to form the primer extension products that are complementary to each CD1d gene sequence nucleic acid strand. Generally, the synthesis will be initiated at the 3′ end of each primer and proceed in the 5′ direction along the template strand, until synthesis terminates, producing molecules of different lengths.

The newly synthesised CD1d strand and its complementary nucleic acid strand will form a double-stranded molecule under hybridizing conditions described above and this hybrid is used in subsequent steps of the process.

The steps of denaturing, annealing, and extension product synthesis can be repeated as often as needed to amplify the target polymorphic gene sequence nucleic acid sequence to the extent necessary. The amount of the specific nucleic acid sequence produced will accumulate in an exponential fashion. This may also be achieved via real time PCR as known in the art.

Preferably, the method of amplifying CD1d is by PCR, as described herein or real time PCR and as is commonly used by those of ordinary skill in the art. Alternative methods of amplification have been described and can also be employed as long as the CD1d sequence amplified by PCR using primers of the invention is similarly amplified by the alternative means. Such alternative amplification systems include but are not limited to self-sustained sequence replication, which begins with a short sequence of RNA of interest and a T7 promoter. Reverse transcriptase copies the RNA into cDNA and degrades the RNA, followed by reverse transcriptase polymerizing a second strand of DNA. Another nucleic acid amplification technique is nucleic acid sequence-based amplification (NASBA) which uses reverse transcription and T7 RNA polymerase and incorporates two primers to target its cycling scheme. NASBA can begin with either DNA or RNA and finish with either, and amplifies to 10⁸ copies within 60 to 90 minutes. Alternatively, nucleic acid can be amplified by ligation activated transcription (LAT). LAT works from a single-stranded template with a single primer that is partially single-stranded and partially double-stranded. Amplification is initiated by ligating a cDNA to the promoter oligonucleotide and within a few hours, amplification is 10⁸ to 10⁹ fold. The QB replicase system can be utilized by attaching an RNA sequence called MDV-1 to RNA complementary to a DNA sequence of interest. Upon mixing with a sample, the hybrid RNA finds its complement among the specimen's mRNAs and binds, activating the replicase to copy the tag-along sequence of interest. Another nucleic acid amplification technique, ligase chain reaction (LCR), works by using two differently labeled halves of a sequence of interest that are covalently bonded by ligase in the presence of the contiguous sequence in a sample, forming a new target. The repair chain reaction (RCR) nucleic acid amplification technique uses two complementary and target-specific oligonucleotide probe pairs, thermostable polymerase and ligase, and DNA nucleotides to geometrically amplify targeted sequences. A 2-base gap separates the oligonucleotide probe pairs, and the RCR fills and joins the gap, mimicking normal DNA repair. Nucleic acid amplification by strand displacement activation (SDA) utilizes a short primer containing a recognition site for hincII with short overhang on the 5′ end that binds to target DNA. A DNA polymerase fills in the part of the primer opposite the overhang with sulfur-containing adenine analogs. HincII is added but only cuts the unmodified DNA strand. A DNA polymerase that lacks 5′ exonuclease activity enters at the site of the nick and begins to polymerize, displacing the initial primer strand downstream and building a new one which serves as more primer. SDA produces greater than 10⁷-fold amplification in 2 hours at 37 degrees C. Unlike PCR and LCR, SDA does not require instrumented temperature cycling. Another amplification system useful in the method of the invention is the QB Replicase System. Although PCR is the preferred method of amplification if the invention, these other methods can also be used to amplify the CD1d sequences as described in the method of the invention.

Polynucleotides of the invention may be incorporated into a recombinant replicable vector for introduction into a host cell. Such vectors may typically comprise a replication system recognized by the host, including the intended polynucleotide encoding the desired polypeptide, and will preferably also include transcription and translational initiation regulatory sequences operably linked to the polypeptide encoding segment. Expression vectors may include, for example, an origin of replication or autonomously replicating sequence (ARS) and expression control sequences, a promoter, an enhancer and necessary processing information sites, such as ribosome-binding sites, RNA splice sites, polyadenylation sites, transcriptional terminator sequences, and mRNA stabilizing sequences. localization signals may also be included where appropriate, which allow the protein to move across cell membranes, and thus attain its functional topology. Such vectors may be prepared by means of standard recombinant techniques well known in the art.

An appropriate promoter and other necessary vector sequences will be selected so as to be functional in the host, and may include, when appropriate, those naturally associated with CD1d genes. Examples of workable combinations of cell lines and expression vectors are known in the art. Many useful vectors are known in the art and may be obtained from such vendors as Stratagene, New England Biolabs, Promega Biotech, and others. Promoters such as the cmv, trp, lac and phage promoters, tRNA promoters and glycolytic enzyme promoters may be used in prokaryotic hosts. Useful yeast promoters include promoter regions for metallothionein, 3-phosphoglycerate kinase or other glycolytic enzymes such as enolase or glyceraldehyde-3-phosphate dehydrogenase, enzymes responsible for maltose and galactose utilization, and others. Vectors and promoters suitable for use in yeast expression are known. Appropriate non-native mammalian promoters might include the early and late promoters from SV40 or promoters derived from murine Moloney leukemia virus, mouse tumour virus, avian sarcoma viruses, adenovirus II, bovine papilloma virus or polyoma. In addition, the construct may be joined to an amplifiable gene (e.g., DHFR) so that multiple copies of the gene may be made. For appropriate enhancer and other expression control sequences.

While such expression vectors may replicate autonomously, they may also replicate by being inserted into the genome of the host cell, by methods well known in the art.

Expression and cloning vectors will likely contain a selectable marker, a gene encoding a protein necessary for survival or growth of a host cell transformed with the vector. The presence of this gene ensures growth of only those host cells that express the inserts. Typical selection genes encode proteins that a) confer resistance to antibiotics or other toxic substances, e.g. ampicillin, neomycin, methotrexate, etc.; b) complement auxotrophic deficiencies, or c) supply critical nutrients not available from complex media, e.g., the gene encoding D-alanine racemase for Bacilli. The choice of the proper selectable marker will depend on the host cell, and appropriate markers for different hosts are well known in the art.

The vectors containing the nucleic acids of interest can be transcribed in vitro, and the resulting RNA introduced into the host cell by well-known methods, e.g., by injection, or the vectors can be introduced directly into host cells by methods well known in the art, which vary depending on the type of cellular host, including electroporation; transfection employing calcium chloride, rubidium chloride, calcium phosphate, DEAE-dextran, or other substances; microprojectile bombardment; lipofection; infection (where the vector is an infectious agent, such as a retroviral genome); and other methods. The introduction of the polynucleotides into the host cell by any method known in the art, including, inter alia, those described above, will be referred to herein as “transformation.” The cells into which have been introduced with nucleic acids described below are meant to also include the progeny of such cells.

### Polynucleotides

An isolated CD1d nucleic acid molecule is disclosed which molecule typically encodes a CD1d polypeptide. The nucleic acid molecule comprises any nucleic acid capable of encoding a functional CD1d polypeptide. Preferably the nucleic acid molecule comprises a nucleic acid capable of encoding a CD1d capable of enhancing an immune response when it is introduced into a DC in a functional immune system. An allelic variant, or analog, including fragments, thereof is also included where it is capable of enhancing an immune response when it is introduced into a DC in a functional immune system. Specifically provided are DNA molecules set out in SEQ ID NOS: 1 or DNA molecules that hybridize to the DNA molecules set out in SEQ ID NOS: 1.

Preferred DNA molecules according to the invention include DNA molecules comprising the sequence set out in SEQ ID NOS: 1.

A polynucleotide is said to “encode” a polypeptide if, in its native state or when manipulated by methods well known to those skilled in the art, it can be transcribed and/or translated to produce the mRNA for and/or the polypeptide or a fragment thereof. The anti-sense strand is the complement of such a nucleic acid, and the encoding sequence can be deduced there-from.

An “isolated” or “substantially pure” nucleic acid (e.g., an RNA, DNA or a mixed polymer) is one which is substantially separated from other cellular components which naturally accompany a native human sequence or protein, e.g., ribosomes, polymerases, many other human genome sequences and proteins. The term embraces a nucleic acid sequence or protein that has been removed from its naturally occurring environment, and includes recombinant or cloned DNA isolates and chemically synthesized analogs or analogs biologically synthesized by heterologous systems.

“CD1d gene sequence,” “CD1d gene,” “CD1d nucleic acids” or “CD1d polynucleotide” each refer to a polynucleotide that contains specific domain such as IgC_MHC_I_alpha3; Class I major histocompatibility complex (MHC) alpha chain immunoglobulin domain. Preferably the nucleic acid molecule comprises a nucleic acid capable of encoding a CD1d, an allelic variant, or analog, including fragments, or mutants thereof. The CD or an allelic variant, or analog, including fragments, or mutants thereof that includes the IgC_MHC_I_alpha3; Class I major histocompatibility complex (MHC) alpha chain immunoglobulin domain. A sequence set out in SEQ ID No. 1.

These terms, when applied to a nucleic acid, refer to a nucleic acid that encodes a CD1d polypeptide, fragment, homologue mutant or variant, including, e.g., protein fusions, mutants or deletions. The nucleic acids of the present invention will possess a sequence that is either derived from, or substantially similar to a natural CD1d encoding gene or one having substantial homology with a natural CD1d encoding gene or a portion thereof. The coding sequences for human CD1d amino acid sequence shown in SEQ ID NOS: 2.

A nucleic acid or fragment thereof is “substantially homologous” (“or substantially similar”) to another if, when optimally aligned (with appropriate nucleotide insertions or deletions) with the other nucleic acid (or its complementary strand), there is nucleotide sequence identity in at least about 60% of the nucleotide bases, usually at least about 70%, more usually at least about 80%, preferably at least about 90%, and more preferably at least about 95-98% of the nucleotide bases. Examples of coding sequence for working substantially homologous fragments are shown in SEQ ID NO: 1, with the amino acid sequence shown in SEQ ID NO: 2.

Alternatively, substantial homology or (identity) exists when a nucleic acid or fragment thereof will hybridise to another nucleic acid (or a complementary strand thereof) under selective hybridisation conditions, to a strand, or to its complement. Selectivity of hybridisation exists when hybridisation that is substantially more selective than total lack of specificity occurs. Typically, selective hybridisation will occur when there is at least about 55% identity over a stretch of at least about 14 nucleotides, preferably at least about 65%, more preferably at least about 75%, and most preferably at least about 90%. The length of homology comparison, as described, may be over longer stretches, and in certain embodiments will often be over a stretch of at least about nine nucleotides, usually at least about 20 nucleotides, more usually at least about 24 nucleotides, typically at least about 28 nucleotides, more typically at least about 32 nucleotides, and preferably at least about 36 or more nucleotides.

Thus, polynucleotides of the invention preferably have at least 75%, more preferably at least 85%, more preferably at least 90% homology to the sequences shown in the sequence listings herein. More preferably there is at least 95%, more preferably at least 98%, homology. Nucleotide homology comparisons may be conducted as described. A preferred sequence comparison program is the GCG Wisconsin Bestfit program. The default scoring matrix has a match value of 10 for each identical nucleotide and −9 for each mismatch. The default gap creation penalty is −50 and the default gap extension penalty is −3 for each nucleotide.

In the context of the present invention, a homologous sequence is taken to include a nucleotide sequence which is at least 60, 70, 80 or 90% identical, preferably at least 95 or 98% identical at the amino acid level over at least 20, 50, 100, 200, 300, 500 or 1000 nucleotides with the nucleotides sequence set out in SEQ ID. NO: 1. In particular, homology should typically be considered with respect to those regions of the sequence that encode contiguous amino acid sequences known to be essential for the function of the protein rather than non-essential neighbouring sequences. Preferred polypeptides of the invention comprise a contiguous sequence having greater than 50, 60 or 70% homology, more preferably greater than 80, 90, 95 or 97% homology, to one or more of the nucleotides sequences encoding polypeptide sequence SEQ ID NO: 2. Preferred nucleic acids preferably contain specific domains as mentioned above.

Nucleotide sequences are preferably at least 15 nucleotides in length, more preferably at least 20, 30, 40, 50, 100 or 200 nucleotides in length. Generally, the shorter the length of the polynucleotide, the greater the homology required to obtain selective hybridization. Consequently, where a polynucleotide of the invention consists of less than about 30 nucleotides, it is preferred that the % identity is greater than 75%, preferably greater than 90% or 95% compared with the CD1d nucleotide sequences set out in the sequence listings herein. Conversely, where a polynucleotide of the invention consists of, for example, greater than 50 or 100 nucleotides, the % identity compared with the CD1d nucleotide sequences set out in the sequence listings herein may be lower, for example greater than 50%, preferably greater than 60 or 75%.

Nucleic acid hybridisation will be affected by such conditions as salt concentration, temperature, or organic solvents, in addition to the base composition, length of the complementary strands, and the number of nucleotide base mismatches between the hybridizing nucleic acids, as will be readily appreciated by those skilled in the art. Stringent temperature conditions will generally include temperatures in excess of 30 degrees C., typically in excess of 37 degrees C., and preferably in excess of 45 degrees C. Stringent salt conditions will ordinarily be less than 1000 mM, typically less than 500 mM, and preferably less than 200 mM. However, the combination of parameters is much more important than the measure of any single parameter. An example of stringent hybridization conditions is 65° C. and 0.1×SSC (1×SSC=0.15 M NaCl, 0.015 M sodium citrate pH 7.0).

The “polynucleotide” compositions of this invention include RNA, cDNA, genomic DNA, synthetic forms, and mixed polymers, both sense and antisense strands, and may be chemically or biochemically modified or may contain non-natural or derivatized nucleotide bases, as will be readily appreciated by those skilled in the art. Such modifications include, for example, labels, methylation, substitution of one or more of the naturally occurring nucleotides with an analog, internucleotide modifications such as uncharged linkages (e.g., methyl phosphonates, phosphotriesters, phosphoamidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, sumoylated site mutants and modified linkages (e.g., alpha anomeric nucleic acids, etc.). Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule.

### Method of Manufacturing Cells for Use in Treatment

Another aspect of the invention includes a method of manufacturing a vaccine for enhancing an immune response to a dendritic cell comprising the steps of:
a) culturing the dendritic cells in vitro;
b) introducing a polynucleotide that encodes a CD1d and overexpresses the CD in the dendritic cell.

In one embodiment the dendritic cell is generated from a human embryonic stem cell. The hESC-DCs may provide a potential and interesting solution to those issues. The hESC-DCs are much cheaper to produce due to the feasibility for large-scale production. They can be functionally defined to ensure therapeutic efficacy via quality control of DC product. The use of hESC-DCs has less logistic issue since the products may go directly from manufacturers into clinics. Moreover, the production of hESC-DCs is scalable to provide unlimited number of DCs, which will benefit patients who require multiple doses of vaccines.

Preferably the polynucleotide is introduced to the dendritic cell in culture by the expression vector as described above.

Another aspect of the invention includes a method of enhancing an immune response to a dendritic cell for treating a patient in need of a cancer treatment comprising the steps of:
a) culturing the dendritic cells in vitro;
b) introducing a polynucleotide that encodes a CD1d and overexpresses the CD1d in the dendritic cell;
c) introducing the CD1d-overexpressing dendritic cell to the patient in need of a cancer treatment.

In one embodiment the dendritic cell is isolated from the patient.

In one embodiment the dendritic cell is generated from a human embryonic stem cell.

An embodiment of the present invention resides in a method for inducing CD1d expression in dendritic cells in vitro in the manufacture of a medicament for treating a patient in need of a cancer treatment comprising the steps of: isolating cells from an individual donor; culturing the cells in vitro; introducing a polynucleotide that encodes CD1d and overexpresses the CD1d in the dendritic cell; introducing the CD1d-overexpressing dendritic cell to the patient in need of a cancer treatment.

“Treatment” and “treat” and synonyms thereof refer to both therapeutic treatment and prophylactic or preventative measures, wherein the object is to prevent or slow down (lessen) a cell proliferative condition. Those in need of such treatment include those already diagnosed with cancer.

As used herein a “therapeutically effective amount” of a compound will be an amount of cells that are capable of preventing or at least slowing down (lessening) a cancerous condition, in particular increasing the lifespan of the patent. Dosages and administration of cells of the invention may be determined by one of ordinary skill in the art. An effective amount of the cells to be employed therapeutically will depend, for example, upon the therapeutic objectives, the route of administration, and the condition of the mammal. Accordingly, it will be necessary for the therapist to adjust the dosage and modify the route of administration as required to obtain the optimal therapeutic effect.

Preferably, the CD1d-overexpressing dendritic cell of the invention is used in cancer such as melanoma, colorectal adenocarcinoma and other tumors.

### Cell Preparation

A “cell”, as used herein, refers to a biological sample obtained from a tissue in the body, or from body fluid. Frequently the cell will be a “clinical sample,” which is a sample derived from a patient such as a fine needle biopsy sample. A “cell” may also include cells isolated from fluids such as blood, serum and the like. Cell samples can be generated from human embryonic stem cells or isolated and obtained from tissues from lung, bladder, brain, uterus, cervix, colon, rectum, esophagus, mouth, head, muscle, heart, skin, kidney, breast, ovary, neck, pancreas, prostate, testis, liver gonads, stomach or from any other organ or tissue known to those skilled in the art.

Cell samples are obtained from the body and include cells and extracellular matter. Cell samples may be from humans or non human animals. Cell samples can be from any organ or fluid. Cell samples can be obtained using known procedures, such as excision, a needle biopsy, blood extraction or the like. The cells are to be processed in a manner that allows culturing and reprogramming of the cells. Accordingly, cells obtained from a subject, donor or individual are ideally washed then immediately cultured.

### Cell Culture

The hESCs may be cultured as known in the art on a relevant culture media such as an artificial medium to grow the cells in vitro for research or medical treatment like Matrigel or mTeSR1. The cells may be passaged though several generations as known in the art to keep the cells continuous. The culture media may contain nutrients to nourish and support the cells. Culture medium may also include growth factors added to produce desired changes in the cells.

Cancer Treatment

Cancer refers to a proliferation of cells such as a tumour. Frequently a tissue sample will be used to verify a patient has cancer and is in need of a treatment as would be known to those skilled in the art. A “clinical sample,” which is a sample derived from a patient such as a fine needle biopsy sample may be used. Tissue samples can be obtained from any tumors such as those of the lung, colon, breast, or cancer tumours located at other sites for example but not limited to bladder, brain, uterus, cervix, colon, rectum, esophagus, mouth, head, skin, kidney, lung, ovary, neck, pancreas, prostate, testis, liver and stomach.

A “tumour” refers to an abnormal growth of tissue that may be comprised of cells that for example, proliferate rapidly. Tumours may be present, for example, in the breast, bladder, brain, uterus, cervix, colon, rectum, esophagus, head, skin, kidney, lung (including Non Small Cell Lung Cancer), ovary, neck, pancreas, prostate, testis, liver and stomach.

The cancer may be treated with a vaccine of the invention comprising a dendritic cell with up-regulated CD1d expression.

In one embodiment the expression vector described above is used for the preparation of a medicament for the treatment of a cancer.

Another aspect of the invention comprises a cancer vaccine comprising a CD1d-overexpressing dendritic cell having upregulated expression of a CD1d polynucleotide.

Preferably the CD1d-overexpressing dendritic cell is selected from a group consisting of a cell isolated from an individual having cancer and a cell generated from a human embryonic stem cell.

Preferably the CD polynucleotide comprises SEQ ID NO. 1.

The term “CD1d-overexpressing dendritic cell” refers to a cell that has a CD expression level higher than an natural human dendritic cell. CD1d expression can be quantified by any means known in the art. Densitomic analysis of western blots, fluorescent antibody tagging such as FACS flow cytometer or other methods known in the art.

Vaccine treatment can be given as a single dose, preferably it is given as at least 2 dose or a plurality of doses. Thus, the present invention also relates to compositions including pharmaceutical compositions comprising a therapeutically effective amount of CD1d-overexpressing dendritic cell. As used herein a compound will be therapeutically effective if it is able to affect tumor growth.

Pharmaceutical forms of the invention suitable for injectable use include sterile aqueous solutions (where water soluble) or dispersions and sterile powders for the extemporaneous preparation of sterile injectable solutions and or one or more carrier. It must be stable under the conditions of manufacture and storage and must be preserved against the contaminating/destructive action of microorganisms such as, for example, bacteria and fungi.

The carrier can be a solvent or dispersion medium containing, for example, buffer solution for maintaining the cells water, ethanol, polyol (for example, glycerol, propylene glycol and liquid polyethylene glycol, and the like), suitable mixtures thereof, and vegetable oils. The proper fluidity can be maintained, for example, by the use of a coating such as, for example, lecithin, by the maintenance of the required particle size in the case of dispersion and by the use of surfactants. Preventing the action of microorganisms in the compositions of the invention is achieved by adding antibacterial and/or antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized active ingredient into a sterile vehicle which contains the basic dispersion medium and the required other ingredients from those enumerated above.

### Examples of Preferred Embodiments

Human embryonic stem cell-derived dendritic cells (hESC-DCs) may potentially provide a platform to generate “off-the-shelf” therapeutic cancer vaccines. To apply the hESC-DCs for cancer immunotherapy in a semiallogeneic setting, it is crucial for these cells to “jump-start” adaptive anti-tumor immunity before their elimination by host alloreaction. Herein, we investigated whether CD up-regulation in hESC-DCs may exploit invariant natural killer T (iNKT) cell adjuvant activity and boost anti-tumor immunity. Using a baculoviral vector carrying the CD gene, we produced CD1d-overexperssing hESC-DCs and demonstrated that the up-regulated CD was functional in presenting α-galactosylceramide (α-GC) for iNKT cell expansion. Pulsed with MART-1 peptide, the CD1d-overexpressing hESC-DCs displayed enhanced capability to prime CD8+ T cells without relying on α-GC-loading. Blocking the CD1d with antibody reduced the immunogenicity, suggesting the importance of hESC-DC and iNKT cell interaction in this context. The CD1d-overexpressing hESC-DCs also induced a pro-inflammatory cytokine profile that may favor the T cell priming. Moreover, similar immunostimulatory effect was observed when the CD1d up-regulation strategy was applied in human monocyte-derived dendritic cells. Therefore, our study suggests that the up-regulation of CD in hESC-DCs provides a novel strategy to enhance their immunogenicity. This approach holds potential for advancing the application of hESC-DCs into human cancer immunotherapy.

Cancer immunotherapy offers great promise in the fight against cancer, especially for persistent malignant cells. The major strength of the approach lies in stimulating the patient's own immune cells to elicit immune responses against tumors. DCs are the most efficient antigen-presenting cells for priming naive T cells into effectors that specifically kill tumors. However, there is an acute need to improve the priming ability in the current DC-based approaches.

We have developed a new technology to enhance the priming ability of DCs by overexpressing CD1d using our in-house developed baculoviral vectors. We demonstrate that CD1d-overexpressing human DCs are effective in improving the activation of invariant natural killer T (iNKT) cells, a type of T cells that function as helper T cells with memory-activated phenotype, which then prime naïve CD8+ T cells against tumor antigen.

We reason that the following factors that are absent in previous DC-based approaches may facilitate the T cell priming by our CD1d-overexpressing human DCs:

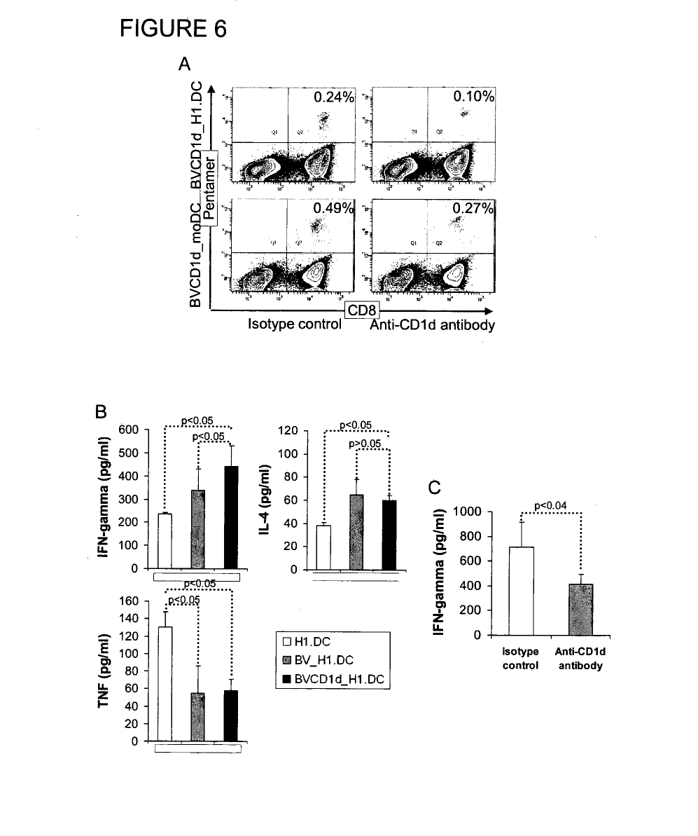

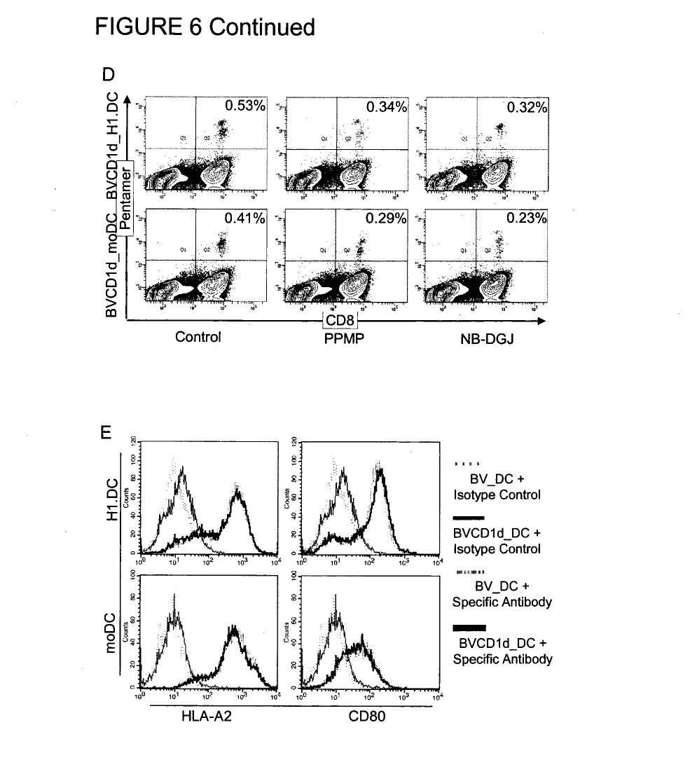

The direct interactions between CD1d-overexpressing DCs and iNKT cells. Pre-treatment with anti-CD1d antibody to block CD1d severely reduced the T cell priming ability of CD1d-overexpressing human DCs (FIG. 6), suggesting the importance of the interaction between DCs and iNKT cells through the binding of overexpressed CD1d to TCR in T cell priming. The mechanism for human system remains unclear, but without being limited to any particular theory it is possible that the DC and iNKT cell interaction somehow licenses the DCs to appropriately activate CD8+ T cells.

The favorable cytokine secretion profile. The cytokine environment may play an important role at the initiation stage of an adaptive immune response. The proper activation of iNKT cells may provide the necessary cytokines to “jump-start” such adaptive immunity. Our result showed CD1d-overexpressing hESC-DCs induced a pro-inflammatory cytokine profile (FIG. 6) that may favor the initiation of antigen-specific T cell response.

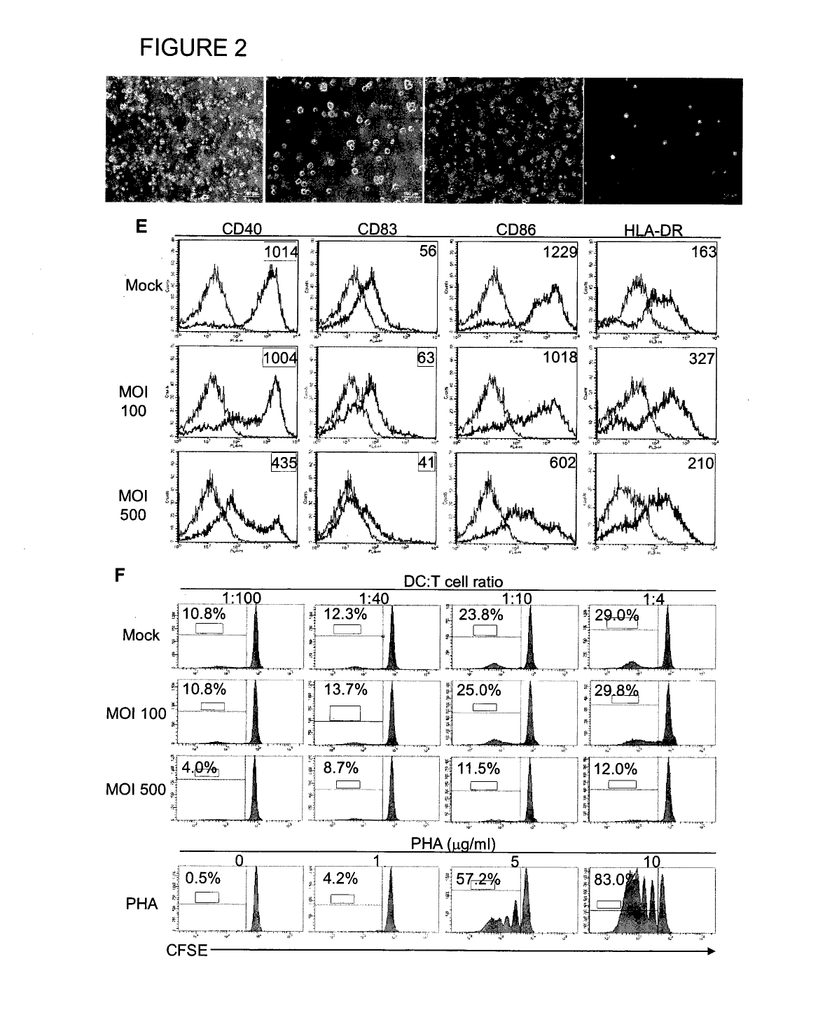

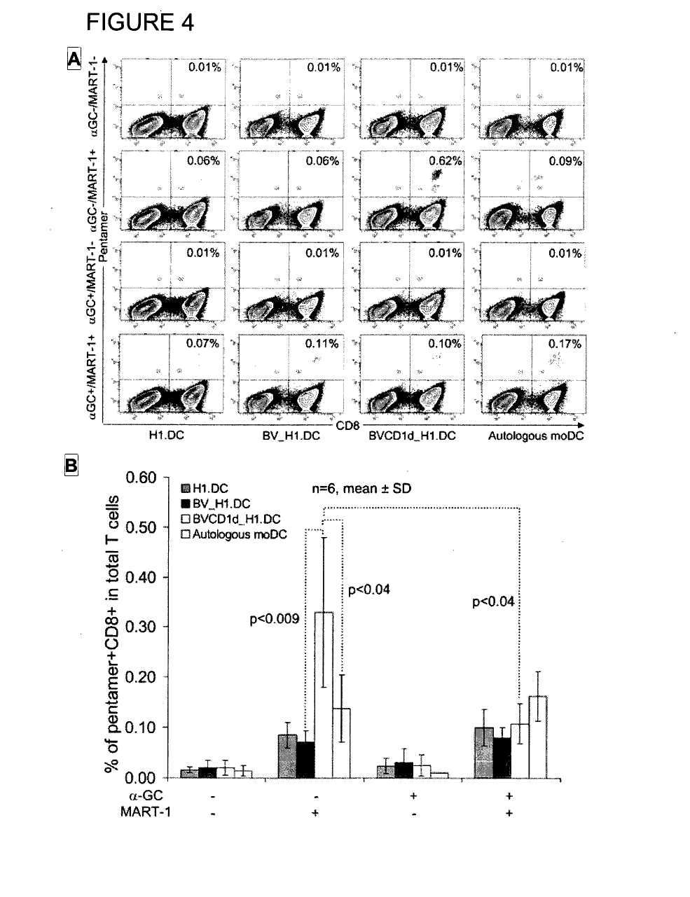

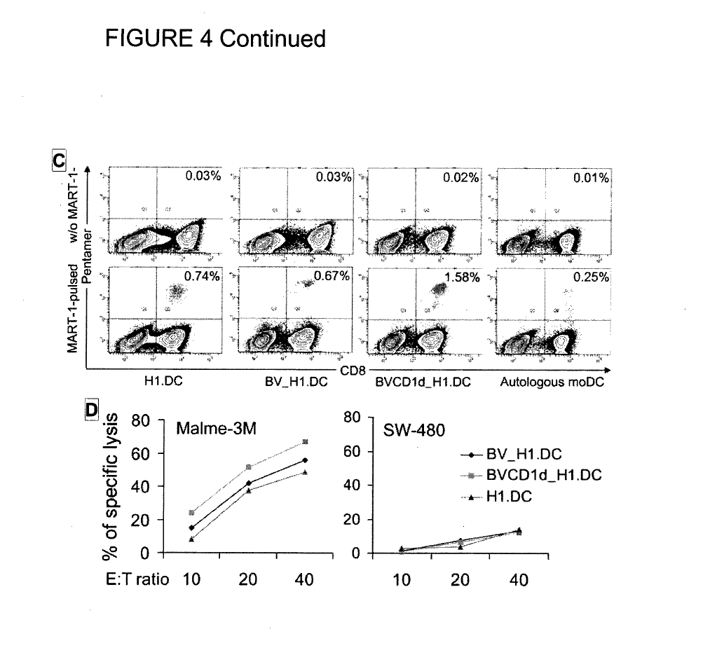

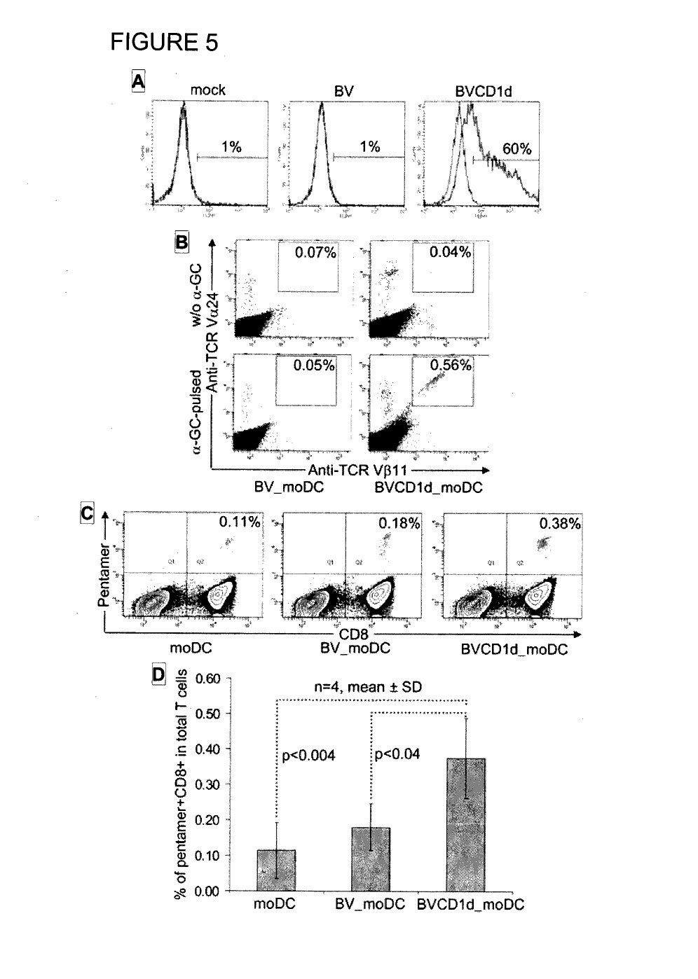

The possible effect of baculoviral transduction on DCs. The baculovirus as a double-stranded-DNA virus may act on myeloid DCs via toll-like receptor-independent pathway and in human moDCs that baculovirus may induce functional maturation. In our study with hESC-DCs, however, we did not observe obvious phenotypic and functional changes after transduction with baculoviral vectors at MOI of 100 (FIG. 2). The baculoviral transduction alone did not enhance the T cell priming ability in hESC-DCs (FIG. 4), though there was slight improvement in moDCs (FIG. 5D), implying that the effect of baculoviral transduction cannot be excluded. Therefore, in our experimental setting, it is likely that the enhanced T cell priming ability of CD1d-overexpressing human DCs is a result of the combined effect of the direct interaction of DCs and iNKT cells, the cytokines produced by iNKT cells through the recognition of endogenous antigen presented on the overexpressed CD1d on DCs, and the possible basal activation via baculoviral transduction.

Our approach not only works well in human embryonic stem cell (hESC)-derived DCs (hESC-DCs), we demonstrate for the first time that anti-tumor immunity of monocyte-derived human DCs can be enhanced as well.

Self-renewing hESCs are inherently immortal, and their proliferation capacity is preserved during long-term cell culture. Hence, they have become a reliable and accessible source of unlimited amounts of uniform human cells. Using hESCs as biological materials to produce cell therapy products enables standardization and large-scale production of cancer immunotherapy vaccines. The advantages over the current DC approaches include:

Produce clinical-grade DC vaccine with consistent phenotype and maturation status. Quality control for each batch of DC product is possible to ensure defined functions and therapeutic efficacy. It would also be possible to employ our increasing understanding of DC biology to enhance DC immunogenicity through genetic modification of hESCs or hESC-derived DCs.

Generate enough cells as a single batch of semi-allo-vaccines for repeated vaccination and for multiple patients, independent of human leukocyte antigen (HLA) haplotype, which may eliminate variability in the quality and composition of the vaccines, and facilitate reliable comparative analysis of clinical outcomes.

Increase cost-effectiveness by eliminating the need for continuous production of tailor-made individual vaccines, simplifying the logistics, and reducing the laboriousness of the vaccine production and delivery process.

Materials and Methods

Cell Culture

A hESC line HES-1 (ES Cell International, Singapore) was maintained on mouse embryonic fibroblasts as described before (23). Two other hESC lines H1 and H9 (WiCell Research Institute, Madison, Wis.) were maintained on feeder-free culture condition on Matrigel (BD Biosciences, San Jose, Calif.)-coated 6-well plate using mTeSR1 medium (STEMCELL Technologies, Vancouver, Canada) according to manufacturer's technical manual. A mouse bone marrow stromal cell line OP9 (ATCC, Manassas, Va.) was maintained with α-MEM (Invitrogen, Carlsbad, Calif.) supplemented with 20% FBS (HyClone, Logan, Utah).

Frozen human PBMCs or HLA-A2+ PBMCs or peripheral blood pan-T cells from healthy donors (STEMCELL Technologies) were thawed and cultured in complete RPMI 1640, which is composed of RPMI 1640 (Invitrogen) supplemented with 10% heat-inactivated human serum AB (Gemini Bio-Products, West Sacramento, Calif.), 2 mM L-glutamine (Invitrogen), 0.1 mM nonessential amino acids (Invitrogen) and 0.1 mM 2-mercaptoethanol (Invitrogen). The human peripheral blood lymphocytes (PBLs) were generated from PBMCs via removing the monocytes by plastic adherence.

A human melanoma cell line Malem-3M (ATCC) was cultured with IMDM (Invitrogen) with 20% FBS. A human colorectal adenocarcinoma cell line SW-480 was maintained in Leibovitz's L-15 Medium (Invitrogen) with 10% FBS.

Generation of Human DCs from hESCs or Monocytes

Human DCs were generated from hESCs based on a three-step protocol as previously described (4). In brief, the hESC aggregates were harvested at the time of subculture and seeded at a density of 2×10⁶ per flask to a T75 flask containing overgrown OP9 cells in α-MEM supplemented with 10% FBS and 100 μM monothioglycerol (Sigma-Aldrich, St Louis, Mo.). The coculture was fed by changing half of the medium every three days. After 9-10 days of coculture, the differentiated hESCs were harvested by enzyme digestion with 1 mg/ml collagenase IV (Invitorgen) and 0.05% trypsin-0.5 mM EDTA (Invitrogen). The cells were then resuspended in α-MEM supplemented with 10% FBS and 100 ng/ml GM-CSF (PeproTech, Rocky Hill, N.J.) and cultured in poly 2-hydroxyethyl methacrylate (Sigma-Aldrich)-coated T75 flask. The suspension culture was fed by changing half of the medium every four days. After 8-10 days, the expanded DC precursors were further differentiated into DCs by culturing in StemSpan serum-free expansion medium (STEMCELL Technologies) supplemented with lipid mixture 1 (Sigma-Aldrich), 100 ng/ml GM-CSF and 100 ng/ml IL-4 (PeproTech) [designated as DC differentiation medium] for 7-9 days with feeding by changing half of the medium every four days. To purify the DCs from the cell culture, the cells were stained with APC mouse anti-human CD209 antibody (BD Biosciences) and sorted using a FACSAria flow cytometer (BD Biosciences). To produce human DCs from monocytes, frozen human PBMCs were thawed and cultured on T75 for 2 h. The plastic-adherent cells were differentiated in DC differentiation medium for 6 days and these monocyte-derived human DCs (moDCs) were then used in the experiments.

Baculovirus Preparation and Transduction of Human DCs

To generate baculoviral vectors carrying the CD1d gene, a transfer plasmid pFastBac1 (Invitrogen) was used. To construct the plasmid, the human cytomegalovirus immediate early gene promoter and enhancer (CMV promoter) was amplified by polymerase chain reaction (PCR) and placed between BamHI and EcoRI cloning sites of pFastBac1. Then, the woodchuck hepatitis virus post-transcriptional regulatory element (WPRE) was PCR-amplified from psubCMV-eGFP-WPRE (kindly provided by Professor H. Büeler, University of Zurich, Switzerland) and inserted between SpeI and XbaI sites of pFastBac 1. The coding sequence of human CD was cloned from pORF9-hCD1D (Invivogen, San Diego, Calif.) through PCR to include the Kozak sequence upstream of its start codon and SalI and SpeI restriction sites at its termini. These two sites were used to insert the CD1d gene. Baculovirus, named as BVCD1d, was produced using the above-described plasmid and propagated in Sf9 cells according to Bac-to-Bac Baculovirus Expression System manual (Invitrogen). A control baculovirus, named as BV, was produced using BacPAK6 viral DNA (Clontech, Mountain View, Calif.) that does not express transgene in mammalian cells. Baculoviruses were purified as described previously (24). Their infectious titers (plaque-forming units, pfu) were determined by plaque assay using Sf9 cells.

For baculoviral transduction, the human DCs were resuspended in DC differentiation medium at a density of 10⁵/100 μl. To transduce the DCs at desired multiplicity of infection (MOI), various numbers of baculoviruses were suspended in 100 μl PBS and added to the cells. After 4 h incubation, the DCs were washed twice and resuspended in DC differentiation medium. Two days after transduction, the eGFP expression was detected under a fluorescence microscope. To detect CD expression, the DCs were stained by PE mouse anti-human CD antibody (BD Biosciences) and analyzed by a FACSCalibur flow cytometer (BD Biosciences).

Measurement of Allostimulatory Function of Human DCs

To measure the allostimulatory function of DCs, frozen human peripheral blood pan-T cells were thawed and used as responders. 10⁷ pan-T cells were first resuspended in 1 ml PBS containing 5% heat-inactivated FBS. Carboxyfluorescein diacetate succinimidyl ester (CFSE; Invitrogen) was added at a final concentration of 0.5 μM to label the T cells in dark at room temperature for 5 min. After washing three times with PBS containing 5% heat-inactivated FBS, the T cells were resuspended in complete RPMI 1640 at a density of 10⁶/ml. To set up allogeneic stimulation assay, 2×10⁵ pan-T cells were co-cultured with graded number of DCs suspended in complete RPMI 1640. A polyclonal activator, phytohemagglutinin (PHA; Sigma-Aldrich) that stimulates T cell proliferation was used as positive control. After incubation of 5 days, the cells were stained with APC mouse anti-human CD3 antibody (BD Biosciences) and the T cell proliferation was measured by CFSE dilution using a FACSAria flow cytometer.

Stimulation and Detection of iNKT Cell Expansion CTL Expansion, CTL Activity and Cytokine Production

To analyze the effect of CD1d-overexpression in DCs on the expansion of human iNKT cells, the hESC-DCs were transduced by BV-CMV.CD1d-WPRE. One day after transduction, the DCs were pulsed with 100 ng/ml α-GC (Axxora, Switzerland) for 24 h. Two days after transduction, 10⁵ α-GC-pulsed DCs were washed and co-cultured with 10⁶ PBL or pan-T cells in complete RPMI 1640 with or without 100 ng/ml α-GC. Seven days after coculture, to detect iNKT cells, the samples were stained with APC mouse anti-human CD3 (BD Biosciences), PE anti-Vα24 TCR (Beckman Coulter, Brea, Calif.) and FITC anti-Vβ11 TCR (Beckman Coulter) and analyzed by a FACSAria flow cytometer.

To stimulate the antigen-specific CTL response, the HLA-A2+H1-derived DCs (H1.DCs) or moDCs were used to present the HLA-A2-restricted epitope MART-1₂₆₋₃₅A27L (ELAGIGILTV; MART-1 peptide; Prolmmune, Oxford, UK). The DCs were first transduced by BV-CMV.CD1d-WPRE and pulsed with or without 100 ng/ml α-GC for 24 h one day after transduction. Two days after transduction, the DCs were pulsed with 10 μg/ml MART-1 peptide for 4 h. After washing, 10⁵ DCs were co-cultured with 10⁶ HLA A2+ PBLs in complete RPMI 1640. After co-culture of 9 days, to detect antigen-specific CTLs, the samples were stained with APC mouse anti-human CD3, FITC-labeled anti-CD8 (ProImmune) and R-PE-labeled A*0201/ELAGIGILTV Pentamer (ProImmune) and analyzed by FACSAria flow cytometer. In some experiments, the CD1d-overexpressing human DCs were pretreated with anti-CD1d blocking antibody (eBioscience, San Diego, Calif.) or its isotype control (eBiosciecne) before applying for T cell priming. To study the effect of glycolipid biosynthesis inhibition on T cell priming, the BVCD1d-transduced human DCs were treated with 20 μM DL-threo-1-Phenyl-2-palmitoylamino-3-morpholino-1-propanol (PPMP; Sigma-Aldrich) or 150 μM N-(n-Butyl)deoxygalactonojirimycin (NB-DGJ; Merck KGaA, Darmstadt, Germany) for 24 hours one day after transduction. In other experiments, the cocultures were restimulated with 10⁵ MART-1 peptide-pulsed DCs on day 9 of coculture and analyzed one week after restimulation. To measure antigen-specific CTL activity, the cocultures were stimulated for the third time on day 7 after second stimulation with 10⁵ MART-1 peptide-pulsed DCs and used as effectors for lysis of tumor cells one week later.

To measure tumor antigen-specific CTL activity, a luminescence-based assay was used. In brief, the tumor cells Malme-3M or SW-480 were labeled with luciferase by transfection with a plasmid containing a luciferase expression cassette using Lipofectamine 2000 (Invitrogen). Two days after transfection, 10⁴ tumor cells were used as targets and incubated with the effectors described above at desired effector to target (E:T) ratios. After 6-hour incubation, D-Luciferin (Caliper Life Sciences, Hopkinton, Mass.) were added to a final concentration of 150 μg/ml. Ten minutes later, luminescence was measured and the percentage of specific lysis was calculated using the relative light unit (RLU) of the experimental sample (with effectors, RLU_{Exp}), the sample with maximum viability (with medium only, RLU_{media}), the sample without viability (with 1% SDS, RLU_{SDS}) as following: % of specific lysis=[1−(RLU_{Exp}−RLU_{SDS})/(RLU_{Media}−RLU_{SDS})]×100.

To study cytokines secreted during co-culture of DCs and PBLs, 10⁵ H1.DCs were transduced by baculoviral vectors. The transduced DCs were then incubated with 10⁶ PBLs in 300 μl complete RPMI 1640. Three days later, the supernatants were collected and cytokine concentration was measured with Cytometric Bead Array (CBA) assay using the CBA Human Soluble Protein Master Buffer Kit and the CBA Flex Sets that detect INF-γ, IL-4 and TNF (BD Biosciences). In some experiments, CD1d-overexpresing H1.DCs were pretreated with anti-CD1d blocking antibody or its isotype control before co-culturing with PBLs.

Results

Generation of Human DCs from hESCs

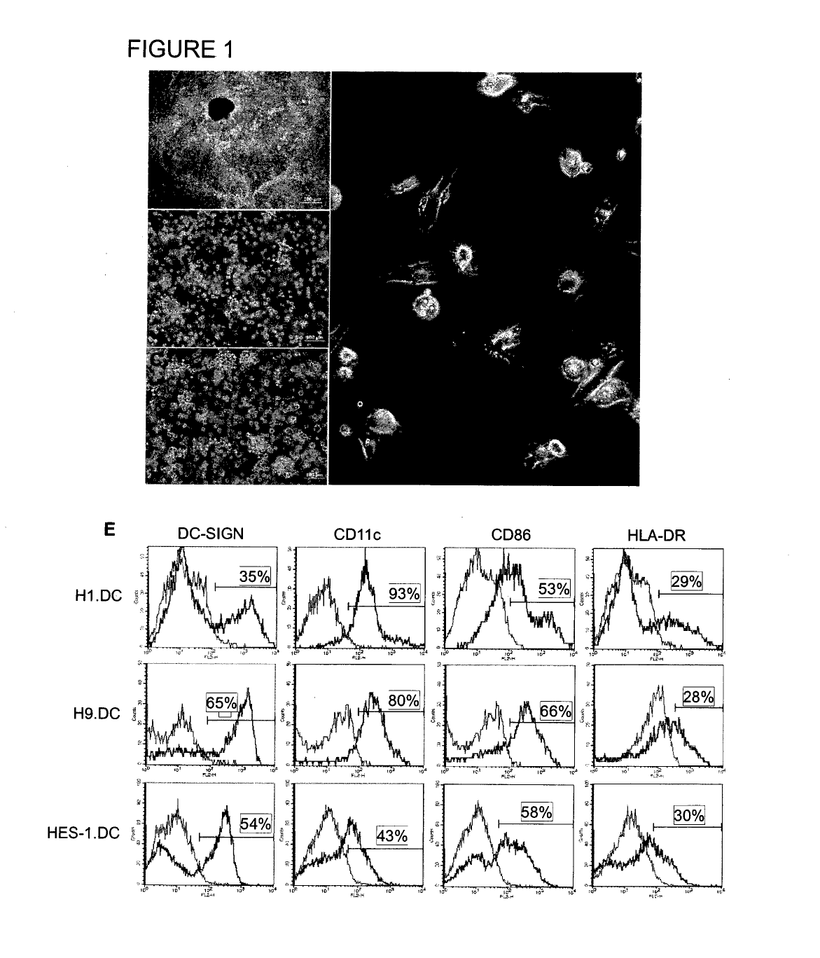

To generate a reliable source of human DCs, a hESC line, HES-1 was used as the starting material. First, the HES-1 cells were induced for myeloid differentiation by coculturing on OP9 cells for 9-10 days (FIG. 1A). To further expand the myeloid lineages, the HES-1/OP9 cocultures were harvested and grown in suspension with GM-CSF for 8-10 days (FIG. 1B). The expanded myeloid cells were then induced to differentiate into DCs by culturing in suspension with GM-CSF and IL-4 for 7-9 days (FIG. 1C). When the cells were grown on normal culture dish, they formed very fine extending dendrites, one of the features that characterizes the DCs (FIG. 1D). Flow cytometery analysis showed that these differentiated HES-1 expressed CD209 (DC-SIGN), CD11c, CD86 and HLA-DR (FIG. 1E), which are typical DC surface markers. To remove the contamination of mouse-derived feeder cells, we also used two other hESC lines that were grown in feeder-free condition to generate human DCs. These two lines H1 and H9 were both able to differentiate into DCs as demonstrated by the expression of surface markers (FIG. 1E), although in our hands H1 line was more reliable and consistent in terms of yield and the H1-derived DCs were used in the following experiments.

Transduction of hESC-DCs by Baculoviral Vectors

To test the feasibility of using baculoviral vector to transduce the hESC-DCs, we first sorted the DCs from the mixed population of differentiated HESCs (FIG. 2A) using FACSAria flow cytometer after staining with APC mouse anti-human CD209 antibody. After sorting the cell population became uniform in size and morphology (FIG. 2B). More importantly, the cells tolerated and survived the sorting condition as demonstrated by the attachment to the normal cell culture dish (FIG. 2C). With these purified hESC-DCs, we tested the transduction with baculoviral vector BV-CMV.eGFP. Live cell fluorescence image shows that using MOI of 100, about 30% of the H1.DC became eGFP+ 2 days after transduction (FIG. 2D), suggesting that baculoviral vectors are able to transduce these DCs.

To understand the effect of baculoviral transduction on phenotype and function of the hESC-DCs, we transduced the H1.DCs at different MOIs. At MOI of 100, the baculoviral transduction did not significantly affect the cell surface expression of CD40, CD83, CD86 and HLA-DR (FIG. 2E). However, at MOI of 500, the expression of these surface molecules was significantly reduced (FIG. 2E). The DC function after baculoviral transduction was further evaluated by their ability to stimulate allogeneic T cells. At MOI of 100, the baculovirus-transduced H1.DC stimulated allogeneic T cell proliferation in a dose-response manner similar to that induced by mock transduced cells (FIG. 2F). Again, at MOI of 500, the allostimulatory function of baculovirus-transduced DCs was obviously affected (FIG. 2F). These results indicate that we may use baculoviral vectors at MOI of 100 for the following transgene functional study, but not at MOI of 500.

CD1d-Overexpression in hESC-DCs Using Baculoviral Vectors

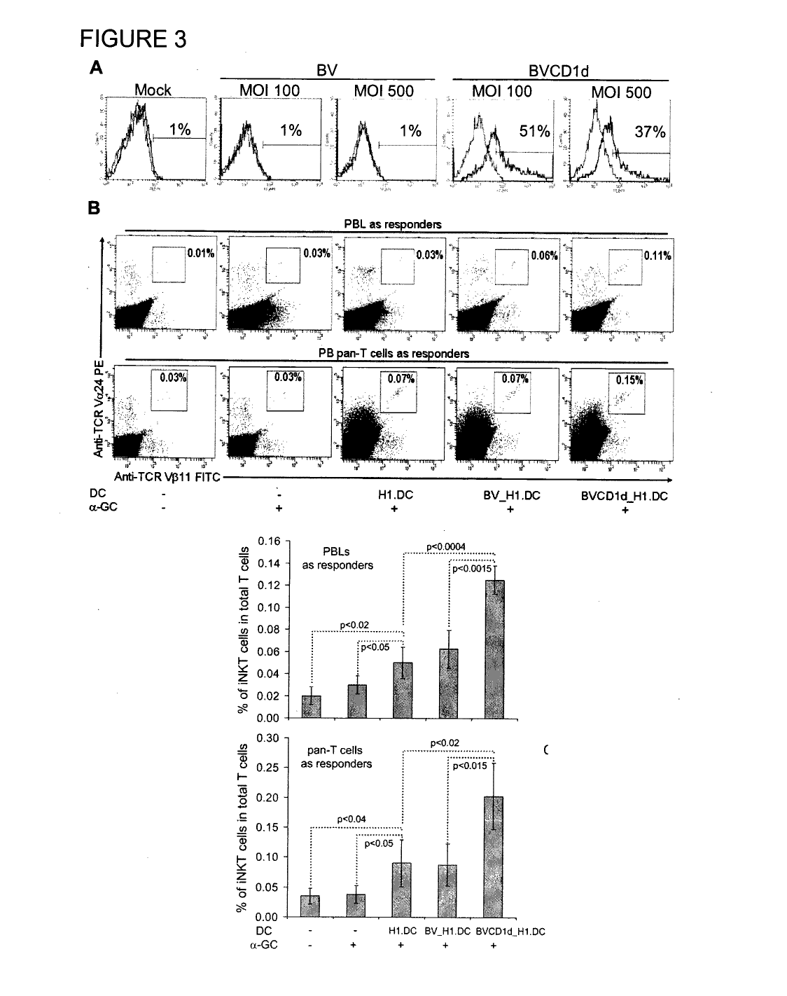

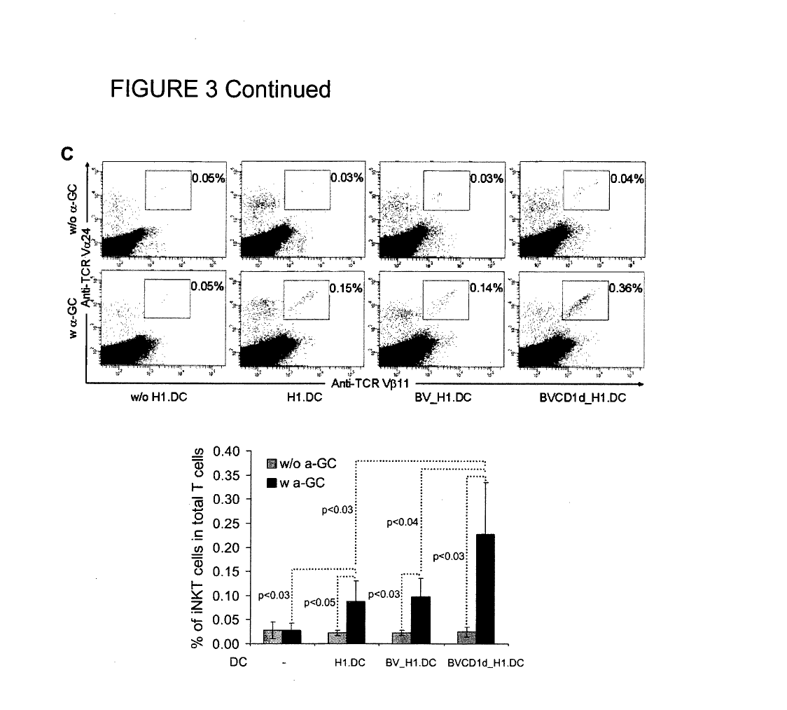

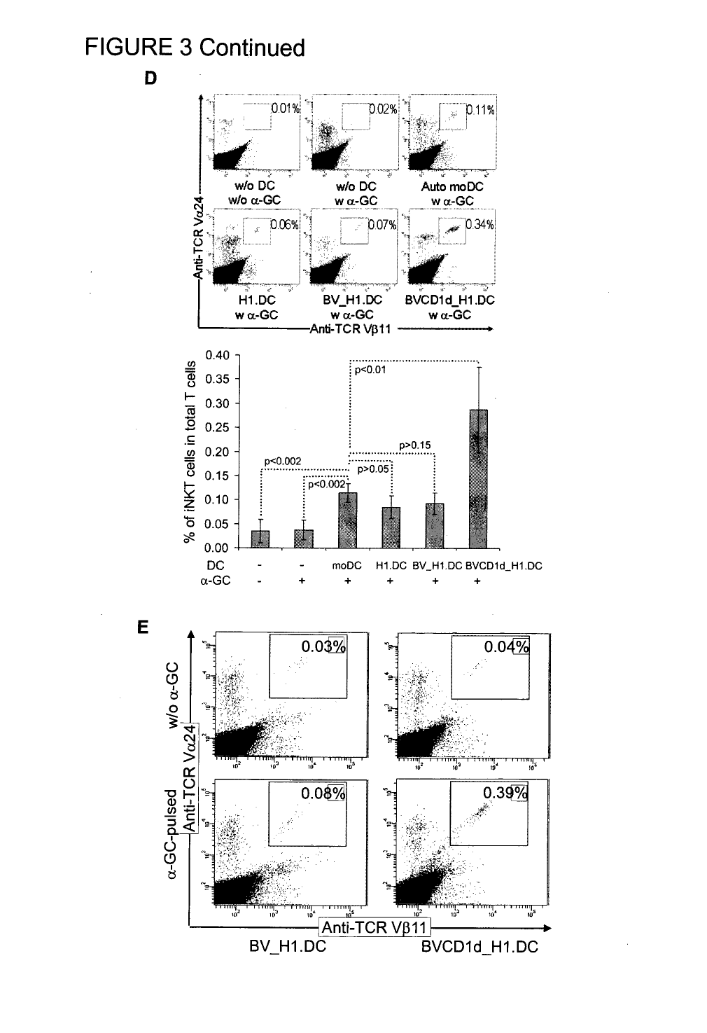

Previously, we showed that the including of WPRE sequence can enhance the baculovirus-mediated transgene expression.^{13,15,16} Here we included the WPRE sequence in BV-CMV.CD1d-WPRE vectors in order to further increase the CD1d expression in hESC-DCs. With this construct, up to 51% of the H1.DCs expressed CD1d 2 days after transduction using MOI of 100 (FIG. 3A). The use of MOI of 500 failed to further increase the CD expression (FIG. 3A). The baculoviral transduction alone did not up-regulate the CD expression in H1.DCs (FIG. 3A).

CD1d Up-Regulation in hESC-DCs is Functional in Promoting Human iNKT Cell Expansion

To study whether these overexpressed CD1d on hESC-DCs are functional, BVCD1d-transduced H1.DCs were evaluated for their capability to present an exogenous glycosphigolipids α-GC for inducing iNKT cell expansion after coculture of 7 days. As shown in FIG. 3B-D, the CD1d-overexpression in H1.DCs enhanced their ability to induce iNKT cell expansion in the presence of α-GC. As shown by using human PBLs as responders, CD1d-overexpressing H1.DCs were more efficient than the mock-transduced H1.DCs and the BV-PacPAK6-transduced H1.DCs for inducing iNKT cell expansion (FIG. 3B). To exclude the possible α-GC presentation by B cells and the residual monocytes in the PBLs, pure human peripheral blood pan-T cells were used as responders and similar result was obtained (FIG. 3B). It was noticeable that the induction of iNKT cell expansion by CD1d-overexpressing H1.DCs depended on the presence of α-GC, while there was no observable cell expansion in the absence of α-GC (FIG. 3C). The efficiency of these CD1d-overexpressing H1.DCs was better than the unmodified autologous human monocyte-derived DCs (FIG. 3D). To exclude the possible effects of free antigen, α-GC-pulsed H1.DCs were used and the result showed that α-GC-pulsed CD1d-overexpressing H1.DCs were good enough for iNKT cell expansion and outperformed those α-GC-pulsed BV-PacPAK6-transduced H1.DCs (FIG. 3E).

CD1d-Overexpression in hESC-DCs Enhances Priming of CD8+ T Cells Against Tumor Antigen

To investigate the effect of the CD1d-overexpression in H1.DCs on their ability to prime antigen-specific CTL response, these modified H1.DCs were pulsed with α-GC and MART-1 peptide and co-cultured with naïve HLA-A2+ PBLs from healthy donors. After one stimulation co-culture for 9 days, the pentamer staining was performed to identify the MART-1 peptide-specific CD8+ T cells (FIGS. 4A and 4B). The result showed that without loading the α-GC, CD1d-overexpression in H1.DCs alone significantly enhanced the expansion of these naïve CD8+ T cells while compared with the BV-PacPAK6-transduced H1.DCs (p<0.009) and the autologous monocyte-derived DCs (p<0.04) (FIG. 4B). On the hand, the loading of α-GC had no obvious beneficial effect in terms of priming these antigen-specific CD8+ T cells by the mock-transduced DCs, the BV-PacPAK6-transduced H1.DCs and the CD1d-overexpressing H1.DCs (FIG. 4B). On the contrary, the priming ability of the CD1d-overexpressing H1.DCs was even significantly decreased with the loading of α-GC comparing with those without α-GC loading (p<0.04) (FIG. 4B). Without loading the MART-1 peptide, there were no observable specific expansion of CD8+ T cells by pentamer staining, suggesting that the expansions were antigen-specific and depended on the presence of MART-1 peptide (FIGS. 4A and 4B) further confirming the specificity of the CD8+ T priming. These results suggest that CD1d-upregulation alone is able to enhance the immunogenicity of the hESC-DCs and this effect does not depend on the loading of exogenous iNKT-cell ligand.

Re-stimulation with MART-1 peptide-loaded H1.DCs further expanded these MART-1 peptide-specific CD8+ T cells and the expansion was more obvious in the coculture initially primed by CD1d-overexpressing H1.DCs (FIG. 4C). Moreover, after third sitmulation with antigen-loaded H1.DCs, these MART-1 peptide-specific CD8+ T cells killed the HLA-A2+ and MART-1+ tumor cell line Malme-3M, but not the HLA-A2+ and MART-1-tumor cell line SW-480 (FIG. 4D), indicating that these T cells were functional and specifically lysed the target tumor cells.

CD1d-Overexpression Strategy can be Applied to moDCs for Enhancing T Cell Priming

Considering the importance of autologous moDCs as one of the useful cell sources in DC-based immunotherapy, we explored the effect of this CD1d-overexpression strategy in moDCs for priming antigen-specific T cell response. After transduction with BV-CMV.CD1d-WPRE at MOI of 100, up to 60% of moDCs expressed CD1d on day 2 (FIG. 5A). Baculoviral transduction alone did not up-regulate the CD expression as demonstrated in moDCs transduced by BV-PacPAK6 (FIG. 5A). Similar to the findings with H1.DCs, α-GC-pulsed CD1d-overexpressing moDCs were obviously superior in expanding the iNKT cells than those α-GC-pulsed BV-PacPAK6-transduced moDCs (FIG. 5B). Without loading the α-GC, CD1d overexpression in moDCs enhanced the priming of MART-1-specific CD8+ T cells (FIGS. 5C and 5D) while comparing with BV-PacPAK6-transduced moDCs (p<0.04) and mock-transduced moDCs (p<0.004). These results suggested that the CD1d-overexpression strategy was not confined to hESC-DCs, but can be extrapolated to moDCs.

Enhancing T Cell Priming by CD1d-Overexpression Depends on DC and iNKT Cell Interaction

To determine whether the stimulatory effect of CD1d-overexpression on the priming of antigen-specific T cells by human DCs is CD1d-restricted, the antigen-loaded CD1d-overexpressing DCs were first blocked with anti-CD1d antibody before applying for T cell priming. In both experiments with CD1d-overexpressing H1.DCs and moDCs, pretreatment with the blocking anti-CD1d antibody decreased the MART-1 peptide-specific CD8+ T cell responses while comparing with pretreatment with isotype control (FIG. 6A). This suggests that the stimulatory effect of CD1d-overexpression on T cell priming is CD1d-dependent and may depend on the interaction of DCs and iNKT cells. To investigate the possible change of cytokine profile that may affect the T cell priming, we measure the concentration of several cytokines in this setting of enhanced DC and iNKT cell interaction due to CD up-regulation. The supernatants from H1.DC and PBL co-cultures were collected on day 3 and analyzed by CBA assay. Although there was some noticeable effect of baculoviral transduction on cytokine production as shown by the reduced TNF production (FIG. 6B), we found that the BVCD1d-transduced H1.DCs induced more pronounced IFN-γ production as compared to the control that used BV-transduced H1.DCs (FIG. 6B). No difference was observed in terms of IL-4 and TNF production between the experiments using BVCD1d-transduced H1.DCs and BV-transduced H1.DCs (FIG. 6B). Pretreating the CD1d-overexpressing H1.DCs with anti-CD1d antibody reduced the IFN-γ production while comparing to the treatment with isotype control (FIG. 6C). These results suggest that CD1d up-regulation may induce a pro-inflammatory cytokine profile that favors T cells priming by DCs.

To evaluate the involvement of endogenous lipid antigen in the stimulatory effect of CD1d up-regulation, the BVCD1d-transduced human DCs were treated with glycolipid biosynthesis inhibitors PPMP or NB-DGJ one day after baculoviral transduction. The results showed that such treatment inhibited the T cell priming ability of CD1d-overexpressing H1.DCs and moDCs (FIG. 6D), suggesting the contribution of endogenous lipid synthesis in the adjuvant effect. Furthermore, to exclude the possible phenotype difference between BV- and BVCD1d-transduced human DCs that may affect the T cell priming ability, the expression levels of HLA-A2 and CD80 were measured two days after transduction. Similar HLA-A2 and CD80 expression levels were observed in BV- and BVCD1d-transduced human DCs (FIG. 6E), which indicates that the stimulatory effect of CD1d up-regulation is not due to the up-regulated expression of HLA class I or costimulatory molecule in BVCD1d-transduced DCs.

Discussion

In the last decade despite the advances in early detection and standard treatment have steadily reduced the death rates in many types of cancers, the high relapse rates in cancer patients remain. This is due to the persistence of malignant cells known as minimal residual disease, which can be not managed by the existing therapies, but poses suitable target for tumor-specific T cells. However, it is still a priority to investigate the strategies that maximize the immunogenicity of DCs to consistently generate maximum anti-tumor immunity, wherein the optimal T cell priming remains an important initial step. Cumulating evidences underscore the importance of interaction between DCs and innate lymphocytes such as natural killer (NK) cells and γδ T cells in the initiation of specific T cell response. DC-NK cell cross talk may induce antitumor CTL in the absence of CD4+ T cells. NK cells may enhance the generation of CD8+ T cell immunity against intracellular parasite. The interaction between DCs and γδ T cells may also help the priming of anti-mycobacterial CD8+ T cell response. These studies suggest the exploitation of these interactions may possibly strengthen the tumor-specific CTL response.

Besides exploiting the adjuvant activity of innate immune system, the exploring of novel dendritic cell source to induce antitumor immune response is equally important. The hESC-DCs may provide a potential and interesting solution to current issues with cancert vaccine. The hESC-DCs are much cheaper to produce due to the feasibility for large-scale production. They can be functionally defined to ensure therapeutic efficacy via quality control of DC product. The use of hESC-DCs has less logistic issue since the products may go directly from manufacturers into clinics. Moreover, the production of hESC-DCs is scalable to provide unlimited number of DCs, which will benefit patients who require multiple doses of vaccines. Therefore, it is of translational significance to investigate novel adjuvant strategies based on such a novel DC source.

iNKT cells are a unique T cell population that lies between innate and adaptive immune system, because they express TCR of the adaptive immune system and act like cells from innate immune system in terms of their specificity and behavior. Once activated, iNKT cells promptly release an array of cytokines that influence many immune cells, thus they are dubbed as the ‘Swiss-Army knife’ of the immune system. Therefore, the optimal activation of iNKT cells that promotes pro-inflammatory instead of tolerizing immune response hold great promise in the development of anti-tumor immunotherapy. Most of the early works in mouse models have focused on the exploitation of the innate functions of iNKT cells using the potent iNKT cell ligand α-GC. One study in mice has first reported that α-GC enhance malaria vaccine-induced protective immunity (29). Another study has demonstrated that α-GC-loaded and peptide antigen-pulsed DCs are more efficient in stimulating CD8+ T cells than peptide-pulsed DCs (30). However, these have failed to enhance T cell priming using α-GC- and OVA peptide-loaded DCs and speculated that it may be due to the elimination of the α-GC- and OVA peptide-loaded DCs by activated iNKT cells or specific CD8+ T cells.²⁴ There were limited studies with human iNKT cells and the results were quite contrasting, which may reflect the differences in the experimental settings and the delicacy of the stimuli that decide the effect of iNKT cells between being pro-inflammatory or anti-inflammatory. In two studies that added free α-GC and antigenic peptide into the coculture of PBMCs and autologous DCs, both groups observed suppressed expansion of antigen-specific CTLs, for which Osada et al and Ho et al attributed it to the Th2 cytokines released by CD4+ iNKT cells and the lysis of APCs or CD1d-bearing activated T cells, respectively.^{25,26} These studies implicate that the use of free α-GC could be an issue in clinical setting, which may be presented not only by DCs, but also other cells like B cells and monocytes that may result in various iNKT cell response and different immunological outcomes. In one study with more clinically-orientated experimental setup, Moreno et al used α-GC and tumor antigen doubled-loaded moDCs to coculture with PBLs and observed that α-GC promoted the CTL priming while using IL-12-overexpressing moDC, but not with unmodified moDCs.²⁷ Therefore, it remains to be understood the exploitation of iNKT cells to enhance priming of anti-tumor CD8+ T cell response.

We reason that one possible explanation for the negative results observed in the above-mentioned human cell studies is the use of α-GC. This potent agonist may overstimulate the human iNKT cells and result in elimination of the α-GC-loaded DCs (16, 17, 31, 34). Fujii et al. have also failed to enhance T cell priming by using the α-GC- and OVA peptide-loaded DCs and they speculated that it may be due to the elimination of such DCs by the activated iNKT cells or specific CD8+ T cells (35). Therefore, overstimulation of iNKT cells could certainly pose a problem for using the α-GC and tumor antigen double-loaded DC strategy to induce anti-tumor immunity, indicating that optimal stimulation of iNKT cells could be critical. The strength of such optimal stimulation may lie between that provided by the physiological level of endogenous iNKT ligands and that rendered by the potent agonist like α-GC. Hence, the up-regulation of antigen/CD1d complexes on the DC surface may provide such optimal strength to activate iNKT cells. To this end, we propose a novel strategy that employs enforced overexpression of CD1d on human DCs to utilize the human iNKT cell adjuvant activity. We demonstrated that such CD1d-overexpressing human DCs were effective in promoting tumor antigen-specific CTL response.

Currently, most of the iNKT cell-based studies have used a potent agonist α-GC as stimulator. It was demonstrated in murine studies that systemic administration of this strong iNKT cell agonist could result in uncontrolled iNKT cell activation and cause side effects such as “cytokine storm” and iNKT cell anergy.^{28,29} The induction of iNKT cell response by α-GC-loaded DCs could reduce these unwanted effects;³⁰ however, the α-GC-loaded DCs could overstimulate iNKT cells and result in lysis of DCs,^{31,32} rendering these DCs unable to initiate a specific protective immune response. It is believed that the amount of signal received by T cells via TCR is crucial in determining the T cell response. One strategy to control the iNKT cell activation is to synthesize α-GC analogs with different length of lipid chain³¹ or modified galactose head grouP³². These structural modifications may fine tune the iNKT TCR affinity of the α-GC analog/CD1d complex and affect the duration of iNKT cell and DC interaction. On the other hand, the concentration of antigen/CD1d complexes on the surface of DCs may also determine the strength of TCR signaling in iNKT cells. In the absence of α-GC, the unpulsed DCs failed to induce iNKT expansion, suggesting that the physiological level of endogenous iNKT ligands are not potent enough to activate iNKT cells.³³ However, in the presence of α-GC, simultaneously loading the DCs with MHC class I binding OVA peptide and α-GC failed to enhance T cell priming, which may be due to the elimination of α-GC-loaded and peptide-pulsed DCs by the expanded and activated iNKT.²⁴ Therefore, providing an optimal stimulation to iNKT cells could be critical for their adjuvant effects on T cell priming and the strength of this stimulation may lie between that provided by physiological level of endogenous iNKT ligands and that rendered by the potent iNKT ligand like α-GC.

In this study, we propose that the overexpression of CD1d on the DC surface may provide an optimal stimulation to iNKT cells and a novel strategy to exploit the adjuvant effect of iNKT cells for T cell priming. To investigate such a hypothesis, we started with the hESC-DCs (FIG. 1), which may not only provide a standardized and unlimited source of human DCs for our experiments but also have great potential for clinical application in immunotherapy. To purposely up-regulate the CD1d expression in the hESC-DCs, baculoviral vectors carrying the CD1d gene were used for transduction (FIG. 3). This baculovirus-mediated CD1d-overexpression was functional as demonstrated by the enhanced stimulation of α-GC-induced iNKT cell expansion (FIG. 3). Interestingly, these CD1d-overexpressing hESC-DCs after pulsing with tumor antigenic peptide did show enhanced capability to prime naïve CD8+ T cells (FIG. 4). Moreover, this CD1d-overexpression strategy can be extrapolated to moDCs as demonstrated by the enhanced T cell priming ability of CD1d-overexpressing moDCs (FIG. 5). We reason that the following factors may facilitate the T cell priming by CD1d-overexpressing human DCs in the absence of α-GC loading: (1) the direct interactions between CD1d-overexpressing DCs and iNKT cells. Pretreatment with anti-CD1d antibody to block CD severely reduced the T cell priming ability of CD1d-overexpressing human DCs (FIG. 6), suggesting the importance of the interaction between DCs and iNKT cells through the binding of overexpressed CD1d to TCR in T cell priming. Although study in murine system showed that the ligation of CD40L on iNKT cells to CD40 on DCs may trigger DCs maturation and induce strong adaptive immune response,³⁴ the mechanism for human system remains unclear, but it is possible that the DC and iNKT cell interaction somehow licenses the DCs to appropriately activate CD8+ T cells. (2) the favorable cytokine secretion profile. The cytokine environment may play an important role at the initiation stage of an adaptive immune response. The proper activation of iNKT cells may provide the necessary cytokines to “jump-start” such adaptive immunity.²⁸ Our result showed CD1d-overexpressing hESC-DCs induced a pro-inflammatory cytokine profile (FIG. 6) that may favor the initiation of antigen-specific T cell response. (3) the possible effect of baculoviral transduction on DCs. It was reported in murine system that baculovirus as a double-stranded-DNA virus may act on myeloid DCs via toll-like receptor-independent pathway³⁵ and in human moDCs that baculovirus may induce functional maturation³⁶. In our study with hESC-DCs, however, we did not observe obvious phenotypic and functional changes after transduction with baculoviral vectors at MOI of 100 (FIG. 2). The baculoviral transduction alone did not enhance the T cell priming ability in hESC-DCs (FIG. 4), though there was slight improvement in moDCs (FIG. 5D), implying that the effect of baculoviral transduction can not be excluded. Therefore, in our experimental setting, it is likely that the enhanced T cell priming ability of CD1d-overexpressing human DCs is a result of the combined effect of the direct interaction of DCs and iNKT cells, the cytokines produced by iNKT cells through the recognition of endogenous antigen presented on the overexpressed CD on DCs and the possible basal activation via baculoviral transduction.

Interestingly, we also observed that the simultaneous loading of α-GC didn't further increase but instead decreased the T cell priming ability of CD1d-overexpressing hESC-DCs (FIG. 4). In the presence of α-GC, there was obvious iNKT cell expansion induced by CD1d-overexpressing hESC-DCs (FIG. 3). These expanded and activated iNKT cells may be detrimental to the DC priming of CTL as previously reported that activated iNKT cells can induce apoptosis in CD40+ DCs.³⁷ Overstimulation of iNKT cells by α-GC may result in iNKT cell anergy^{29,38} as well as lysis of α-GC-loaded APCs via TCR engagement.^{31,32} It was reported that the NK:DC ratios may be important for the immunological outcome for DC-NK cell interaction, wherein low NK:DC ratios resulting in DC maturation and high NK:DC ratios resulting in DC death.³⁹ Similarly, these effects may need to be considered during DC-iNKT cell interaction, wherein excessive expansion of iNKT cells may result in the elimination of antigen-presenting DCs and the subsequent T cell response. Our finding here further emphasizes the importance of the optimal activation of iNKT cells for enhanced T cell priming.

Overall, this study has demonstrated that the enforced expression of CD1d on hESC-DCs and monocyte-derived DCs enhanced the DC efficacy in priming CD8+ T cells against tumor antigen. The easy process to generate large amount of uniform hESC-DCs, their genetic manipulability, and their competence in inducing anti-tumor immunity indicate that the hESC-DCs can potentially be used as an unlimited cell source to produce “off-the-shelf” DC-based vaccines.

Those skilled in the art will appreciate that the invention described herein is susceptible to variations and modifications other than those specifically described. The invention includes all such variation and modifications. The invention also includes all of the steps, features, formulations and compounds referred to or indicated in the specification, individually or collectively and any and all combinations or any two or more of the steps or features.

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness.

Any manufacturer's instructions, descriptions, product specifications, and product sheets for any products mentioned herein or in any document incorporated by reference herein, are hereby incorporated herein by reference, and may be employed in the practice of the invention.

The present invention is not to be limited in scope by any of the specific embodiments described herein. These embodiments are intended for the purpose of exemplification only. Functionally equivalent products, formulations and methods are clearly within the scope of the invention as described herein.

The invention described herein may include one or more range of values (eg size, concentration etc). A range of values will be understood to include all values within the range, including the values defining the range, and values adjacent to the range which lead to the same or substantially the same outcome as the values immediately adjacent to that value which defines the boundary to the range.

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

While the invention has been described with reference to specific methods and embodiments, it will be appreciated that various modifications and changes may be made without departing from the invention.

### REFERENCES

1. Gilboa E. DC-based cancer vaccines. J Clin Invest. 2007; 117:1195-1203.
2. DeFrancesco, L. 2010. Landmark approval for Dendreon's cancer vaccine. Nat Biotechnol 28:531-532.
3. Senju, S., H. Suemori, H. Zembutsu, Y. Uemura, S. Hirata, D. Fukuma, H. Matsuyoshi, M. Shimomura, M. Haruta, S. Fukushima, Y. Matsunaga, T. Katagiri, Y. Nakamura, M. Furuya, N. Nakatsuji, and Y. Nishimura. 2007. Genetically manipulated human embryonic stem cell-derived dendritic cells with immune regulatory function. Stem Cells 25:2720-2729.
4. Slukvin, II, Vodyanik M A, Thomson J A, Gumenyuk M E, Choi K D. Directed differentiation of human embryonic stem cells into functional dendritic cells through the myeloid pathway. J. Immunol. 2006; 176:2924-2932.
5. Su, Z., C. Frye, K. M. Bae, V. Kelley, and J. Vieweg. 2008. Differentiation of human embryonic stem cells into immunostimulatory dendritic cells under feeder-free culture conditions. Clin Cancer Res 14:6207-6217.
6. Reschner A, Hubert P, Delvenne P, Boniver J, Jacobs N. Innate lymphocyte and dendritic cell cross-talk: a key factor in the regulation of the immune response. Clin Exp Immunol. 2008; 152:219-226.
7. Bendelac A, Savage P B, Teyton L. The biology of NKT cells. Annu Rev Immunol. 2007; 25:297-336.
8. Niemeyer M, Darmoise A, Mollenkopf H J, et al. Natural killer T-cell characterization through gene expression profiling: an account of versatility bridging T helper type 1 (Th1), Th2 and Th17 immune responses. Immunology. 2008; 123:45-56.
9. Rolf J, Berntman E, Stenstrom E. M. Smith, R. Mansson, H. Stenstad, T. Yamagata, W. Agace, M. Sigvardsson, and S. L. Cardell.2008. Molecular profiling reveals distinct functional attributes of CD1d-restricted natural killer (NK) T cell subsets. Mol. Immunol.; 45:2607-2620.
10. Metelitsa L S, Weinberg K I, Emanuel P D, Seeger R C. Expression of CD1d by myelomonocytic leukemias provides a target for cytotoxic NKT cells. Leukemia. 2003; 17:1068-1077.
11. Nieda M, Nicol A, Koezuka Y, Koezuka, A. Kikuchi, N. Lapteva, Y. Tanaka, K. Tokunaga, K. Suzuki, N. Kayagaki, H. Yagita, H. Hirai, and T. Juji. 2001. TRAIL expression by activated human CD4(+)V alpha 24NKT cells induces in vitro and in vivo apoptosis of human acute myeloid leukemia cells. Blood.; 97:2067-2074.
12. Metelitsa L S, Naidenko O V, Kant A, H. W. Wu, M. J. Loza, B. Perussia, M. Kronenberg, and R. C. Seeger. 2001. Human NKT cells mediate antitumor cytotoxicity directly by recognizing target cell CD1d with bound ligand or indirectly by producing IL-2 to activate NK cells. J. Immunol.; 167:3114-3122.
13. Cerundolo, V., J. D. Silk, S. H. Masri, and M. Salio. 2009. Harnessing invariant NKT cells in vaccination strategies. Nat Rev Immunol 9:28-38.
14. Parekh, V. V., M. T. Wilson, D. Olivares-Villagomez, A. K. Singh, L. Wu, C. R. Wang, S. Joyce, and L. Van Kaer. 2005. Glycolipid antigen induces long-term natural killer T cell anergy in mice. J Clin Invest 115:2572-2583.
15. Fujii, S., K. Shimizu, M. Kronenberg, and R. M. Steinman. 2002. Prolonged IFN-gamma-producing NKT response induced with alpha-galactosylceramide-loaded DCs. Nat Immunol 3:867-874.
16. McCarthy, C., D. Shepherd, S. Fleire, V. S. Stronge, M. Koch, P. A. Illarionov, G. Bossi, M. Salio, G. Denkberg, F. Reddington, A. Tarlton, B. G. Reddy, R. R. Schmidt, Y. Reiter, G. M. Griffiths, P. A. van der Merwe, G. S. Besra, E. Y. Jones, F. D. Batista, and V. Cerundolo. 2007. The length of lipids bound to human CD1d molecules modulates the affinity of NKT cell TCR and the threshold of NKT cell activation. J Exp Med 204:1131-1144.
17. Silk, J. D., M. Salio, B. G. Reddy, D. Shepherd, U. Gileadi, J. Brown, S. H. Masri, P. Polzella, G. Ritter, G. S. Besra, E. Y. Jones, R. R. Schmidt, and V. Cerundolo. 2008. Cutting edge: nonglycosidic CD lipid ligands activate human and murine invariant NKT cells. J Immunol 180:6452-6456.
18. Venkataswamy, M. M., and S. A. Porcelli. 2010. Lipid and glycolipid antigens of CD1d-restricted natural killer T cells. Semin Immunol 22:68-78.
19. Skold, M., X. Xiong, P. A. Illarionov, G. S. Besra, and S. M. Behar. 2005. Interplay of cytokines and microbial signals in regulation of CD1d expression and NKT cell activation. J Immunol 175:3584-3593.
20. Hage, C. A., L. L. Kohli, S. Cho, R. R. Brutkiewicz, H. L. Twigg, 3rd, and K. S. Knox. 2005. Human immunodeficiency virus gp120 downregulates CD cell surface expression. Immunol Lett 98:131-135.
21. Webb, T. J., R. A. Litavecz, M. A. Khan, W. Du, J. Gervay-Hague, G. J. Renukaradhya, and R. R. Brutkiewicz. 2006. Inhibition of CD1d1-mediated antigen presentation by the vaccinia virus B1R and H5R molecules. Eur J Immunol 36:2595-2600.
22. Yuan, W., A. Dasgupta, and P. Cresswell. 2006. Herpes simplex virus evades natural killer T cell recognition by suppressing CD recycling. Nat Immunol 7:835-842.
23. Zeng, J., J. Du, Y. Zhao, N. Palanisamy, and S. Wang. 2007. Baculoviral vector-mediated transient and stable transgene expression in human embryonic stem cells. Stem Cells 25:1055-1061.
24. Zeng, J., J. Du, J. Lin, X. Y. Bak, C. Wu, and S. Wang. 2009. High-efficiency transient transduction of human embryonic stem cell-derived neurons with baculoviral vectors. Mol Ther 17:1585-1593.
25. Fabre, J. W. 2001. The allogeneic response and tumor immunity. Nat Med 7:649-652.
26. de Gruijl, T. D., A. J. van den Eertwegh, H. M. Pinedo, and R. J. Scheper. 2008. Whole-cell cancer vaccination: from autologous to allogeneic tumor- and dendritic cell-based vaccines. Cancer Immunol Immunother 57:1569-1577.
27. Fujii, S., K. Liu, C. Smith, A. J. Bonito, and R. M. Steinman. 2004. The linkage of innate to adaptive immunity via maturing dendritic cells in vivo requires CD40 ligation in addition to antigen presentation and CD80/86 costimulation. J Exp Med 199:1607-1618.
28. Fujii, S., K. Shimizu, C. Smith, L. Bonifaz, and R. M. Steinman. 2003. Activation of natural killer T cells by alpha-galactosylceramide rapidly induces the full maturation of dendritic cells in vivo and thereby acts as an adjuvant for combined CD4 and CD8 T cell immunity to a coadministered protein. J Exp Med 198:267-279.
29. Gonzalez-Aseguinolaza, G., L. Van Kaer, C. C. Bergmann, J. M. Wilson, J. Schmieg, M. Kronenberg, T. Nakayama, M. Taniguchi, Y. Koezuka, and M. Tsuji. 2002. Natural killer T cell ligand alpha-galactosylceramide enhances protective immunity induced by malaria vaccines. J Exp Med 195:617-624.
30. Stober, D., I. Jomantaite, R. Schirmbeck, and J. Reimann. 2003. NKT cells provide help for dendritic cell-dependent priming of MHC class I-restricted CD8+ T cells in vivo. J Immunol 170:2540-2548.
31. Ho, L. P., B. C. Urban, L. Jones, G. S. Ogg, and A. J. McMichael. 2004. CD4(−) CD8alphaalpha subset of CD1d-restricted NKT cells controls T cell expansion. J Immunol 172:7350-7358.
32. Osada, T., M. A. Morse, H. K. Lyerly, and T. M. Clay. 2005. Ex vivo expanded human CD4+regulatory NKT cells suppress expansion of tumor antigen-specific CTLs. Int Immunol 17:1143-1155.
33. Moreno, M., J. W. Molling, S. von Mensdorff-Pouilly, R. H. Verheijen, E. Hooijberg, D. Kramer, A. W. Reurs, A. J. van den Eertwegh, B. M. von Blomberg, R. J. Scheper, and H. J. Bontkes. 2008. IFN-gamma-producing human invariant NKT cells promote tumor-associated antigen-specific cytotoxic T cell responses. J Immunol 181:2446-2454.
34. Nieda, M., A. Kikuchi, A. nicol, Y. Koezuka, Y. Ando, S. Ishihara, N. Lapteva, T. Yabe, K. Tokunaga, K. Tadokoro, and T. Juji. 2001. Dendritic cells rapidly undergo apoptosis in vitro following culture with activated CD4+Valpha24 natural killer T cells expressing CD40L. Immunology 102:137-145.
35. Fujii, S., S. Motohashi, K. Shimizu, T. Nakayama, Y. Yoshiga, and M. Taniguchi. 2010. Adjuvant activity mediated by iNKT cells. Semin Immunol 22:97-102.
36. Abe, T., Y. Kaname, X. Wen, H. Tani, K. Moriishi, S. Uematsu, O. Takeuchi, K. J. Ishii, T. Kawai, S. Akira, and Y. Matsuura. 2009. Baculovirus induces type I interferon production through toll-like receptor-dependent and -independent pathways in a cell-type-specific manner. J Virol 83:7629-7640.
37. Schutz, A., N. Scheller, T. Breinig, and A. Meyerhans. 2006. The Autographa californica nuclear polyhedrosis virus AcNPV induces functional maturation of human monocyte-derived dendritic cells. Vaccine 24:7190-7196.

1. An expression vector comprising a polynucleotide that encodes a CD1d operably linked to a regulatory sequence capable of up-regulating expression of said polynucleotide in a host cell.

2. The expression vector of claim 1 wherein the polynucleotide encodes a CD1d comprising SEQ ID NO. 1.

3. The expression vector of claim 1 for use in the manufacture of a vaccine for the treatment of a cancer.

4. The expression vector of claim 1 wherein the vector is a baculoviral vector carrying the CD1d polypeptide.

5. The expression vector of claim 1 wherein the vector is able to stably or transiently express the CD1d in the dendritic cells or a progenitor cell.

6. The expression vector of claim 1 wherein the host cell is a dendritic cell or a progenitor cell such as a human embryonic stem cell or a human induced pluripotent stem cell.

7. The expression vector of claim 6 wherein the dendritic cell is derived from a human embryonic stem cell or a human pluripotent stem cell.

8. A method of manufacturing a vaccine for enhancing an immune response to a dendritic cell comprising:
a) culturing the dendritic cells in vitro;
b) introducing a polynucleotide that encodes a CD1d and overexpresses the CD1d in the dentritic cell.

9. The method of claim 8 wherein the polynucleotide comprises SEQ ID NO. 1.

10. The method of claim 8 wherein the dendritic cell is generated from a human embryonic stem cell or a human pluripotent stem cell.

11. A method of manufacturing a vaccine for enhancing an immune response to a dendritic cell comprising:
a) culturing the dendritic cells in vitro;
b) introducing a polynucleotide that encodes a CD1d and overexpresses the CD1d in the dentritic cell, wherein the polynucleotide is introduced to the dendritic cell in culture by an expression vector comprising a polynucleotide that encodes a CD1d operably linked to a regulatory sequence capable of up-regulating expression of said polynucleotide in a host cell.

12. A method of enhancing an immune response to a dendritic cell for treating a patient in need of a cancer treatment comprising:
a) culturing the dendritic cells in vitro;
b) introducing a polynucleotide that encodes a CD1d and overexpresses the CD1d in the dendritic cell;
c) introducing the CD1d-overexpressing dendritic cell to the patient in need of a cancer treatment.

13. The method of claim 12 wherein the dendritic cell is isolated from the patient.

14. The method of claim 12 wherein the dendritic cell is generated from a human embryonic stem cell or a human pluripotent stem cell.

15. The method of claim 11 wherein the polynucleotide comprises SEQ ID NO. 1.

16. A use of an expression vector comprising a polynucleotide that encodes a CD1d operably linked to a regulatory sequence capable of up-regulating expression of said polynucleotide in a host cell, for the preparation of a medicament for the treatment of a cancer.

17. A cancer vaccine comprising a CD1d-overexpressing dendritic cell having upregulated expression of a CD1d polynucleotide.

18. The cancer vaccine of claim 17 wherein the dendritic cell is selected from a group consisting of a cell isolated from an individual having cancer and a cell generated from a human embryonic stem cell or a human pluripotent stem cell.

19. The cancer vaccine of claim 17 wherein the CD1d polynucleotide comprises SEQ ID NO. 1.
